(19) United States
(12) Patent Application Publication
(10) Pub. No.: US 2011/0157588 A1
(43) Pub. Date: Jun. 30, 2011

(54) DEVICE COMPRISING TWO REFLECTING LAYERS AND A POLYMER LAYER FOR ANALYZING THE AGE AND/OR QUALITY OF A NATURAL PRODUCT

(75) Inventors: Fritz Pittner, Wien (AT); Maria Bauer, Salzburg (AT); Georg Bauer, Salzburg (AT); Ralph Domnick, Buckenhof (DE); Nadira Ibrisimovic, Wien (AT)

(73) Assignees: UNIVERSITÄT WIEN, Wien (AT); MAX F. PERUTZ LABORATORIES GMBH, Wien (AT)

(21) Appl. No.: 12/747,835

(22) PCT Filed: Dec. 12, 2008
(86) PCT No.: PCT/EP2008/067423

§ 371 Date: Feb. 21, 2011

(30) Foreign Application Priority Data
Dec. 13, 2007 (EP) PCT/EP2007/063895

(51) Int. Cl. G01J 3/46; G01N 33/52; B05D 5/06; B82Y 30/00

(52) U.S. Cl. 356/402; 422/400; 427/162; 977/773

(57) ABSTRACT

The present invention relates to the field of analyzing the age and/or quality of certain natural products, for example foods. The invention also relates to devices for analyzing said age and/or quality as well as to methods for preparing such devices, to methods for analyzing natural products and to their use.

### FIELD OF THE INVENTION

The present invention relates to the field of optical sensors for analyzing the age and/or quality of a natural product comprising foods and cosmetic products.

In this context, the invention describes devices comprising two reflecting layers and a polymer layer positioned between said two reflecting layers, methods for preparing such devices as well as the use of such devices.

The invention further describes devices with a reflecting layer with a refractive index n1, a transparent layer with a refractive index n2, a polymer layer with a refractive index n3 and a semi-reflecting layer with a refractive index n4, wherein n2 is >n1, n3 and n4.

### BACKGROUND OF THE INVENTION

Control of the quality of perishable foods and/or cosmetic products is a critical task throughout the storage, production, distribution and consumption/use of such natural products.

Many foods are subject to spoilage, which may be caused by improper production and/or handling and/or storage. If, for example, perishable products such as milk or meat are exposed to excessive temperatures during transport, they will age and spoil prematurely.

Ultimately, aging processes will lead to spoilage of most if not all foods even if the right handling and storage conditions were chosen. Aging processes leading to the spoilage of foods are caused in most cases by microorganisms. Examples for the spoilage of foods caused by microorganisms comprise: the spoilage of fish and meat caused by the infestation with e.g. Acetinobacter, Moraxella, Pseudomonas species, Lactobacillus species, Bacillus sp., Micrococcus, Clastridium botulinum, Salmonella species, Listeria species; the spoilage of milk products caused by the infestation with lactic acid bacteria; the spoilage of foods containing carbohydrates such as bread caused by molds.

In general, the shelf life and thus the freshness/quality of foods are estimated by an expiry date. However, this method is inaccurate for several reasons, one being that the actual number of microorganisms/bacteria on foods is typically unknown. Another reason may be improper handling as mentioned above. On the other hand, it is also the case that foods still being in good condition and of good quality are discarded only because of their expiry date has expired.

Spoiled foods can be regarded as major source of diseases. In the U.S. alone there are 76 million cases of food-borne illnesses annually. Furthermore, foods spoiled, e.g. because of incorrect handling and/or storage represent major lost revenue for companies. As mentioned above, some foods are discarded without justification, which again is lost economical revenue.

Due to enclosing many foods into packaging, it may not be readily apparent when the product has exceeded its lifetime for consumption. Tests and experiments performed in laboratories to monitor the quality and age of such foods are usually very time-consuming and can neither be performed en-route nor directly.

To ease quality control of foods and cosmetic products, there is a need for a device that cheaply, rapidly and easy visibly indicates the condition of such products. Such a device should give information about the condition/quality of the natural product and/or microbial contamination during handling and storage periods.

### OBJECTS AND SUMMARY OF THE INVENTION

It is an objective of the present invention to provide a device which can be used to analyze the age and/or quality of natural products, e.g. foods.

It is yet another objective of the present invention to provide a method for preparing such a device.

It is a further objective of the present invention to provide a method for analyzing the age and/or quality of a natural product with said device.

It is an objective of the present invention to describe the use of said device for the analysis of the age and/or quality of a natural product.

These and other objectives of the present invention, as they will become apparent from the ensuing description, are solved by the subject matter of the independent claims. The dependent claims relate to some of the preferred embodiments of the invention.

According to a first aspect of the invention, a device for analyzing the age and/or quality of a natural product is provided comprising two reflecting layers and a polymer layer positioned between said two reflecting layers. Said device is configured in such a way that biomolecules are allowed to penetrate at least one of said reflecting layers in order to contact said polymer layer. Furthermore, said device is configured in such a way that a change in the thickness and/or the refraction index of said polymer layer results in a colour change visible to the human eye. Typically, one of the two reflecting layer will have a small thickness than the other layer in order to act as a semi-reflecting layer.

In a preferred embodiment of this first aspect of the present invention, at least one reflecting layer has a thickness of 1 to 100 nm. In a further preferred embodiment of the present invention, the polymer layer has a thickness of 5 to 1000 nm prior to being contacted by said biomolecules.

In a further preferred embodiment of this first aspect of the invention, at least one reflecting layer comprises a mirror layer made of an electrically conductive material. In yet another embodiment of the present invention, said reflecting/mirror layer is made of an electrically conductive metal or an electrically conductive metal film. In a further preferred embodiment, said mirror layer is made of gold or a gold film. In a preferred embodiment of the present invention, the reflecting/mirror layer made of gold has a thickness of 10 to 30 nm. In an also preferred embodiment, said mirror layer is made of titan or a titan film. In a preferred embodiment of the present invention, the reflecting/mirror layer made of titan has a thickness of 10 to 60 nm.

In another preferred embodiment of this first aspect of the present invention, the two reflecting layers are identical. In yet another preferred embodiment of the present invention, said two reflecting layers differ with respect to their thickness and/or their material/composition.

In a further preferred embodiment of the first aspect of the present invention, the device described above comprises an additional layer between the two reflecting layers apart from the polymer layer. Said additional layer has a high refractive index. It can be made of TiO₂. Said additional layer may have a thickness of 5 to 150 nm.

A second aspect of the present invention relates to a device comprising at least
a. a first reflecting layers with a refractive index n1;
b. a second transparent layer with a refractive index n2 positioned above said first reflecting layer;
c. a third polymer with a refractive index n3 positioned above said second transparent layer;
d. a fourth semi-reflecting layer with a refractive index n4 positioned above said third polymer layer;
wherein the device is configured in such a way that biomolecules are allowed to penetrate at least the fourth semi-reflecting layer in order to contact said third polymer layer,
wherein the device is configured in such a way that a change in the thickness and/or the refraction index of said third polymer layer results in a colour change visible to the human eye; and wherein the n2 is >n1, n3 and n4.

As far as the first reflecting layer and the third polymer layer of this second aspect of the invention is concerned, all comments made above on the properties, composition and/or thickness of the reflecting layer and polymer layer of the first aspect of the invention equally applies.

Thus, the first reflecting layer may have a thickness of about 1 to about 100 nm, of about 5 to about 90 nm, of about 10 to about 80 nm such as about 20 nm, about 30 nm, about 40 nm, about 50 nm, about 60 nm or about 70 nm. Typically, the first reflecting layer will be a continuous layer. In a further preferred embodiment, said first reflecting layer may comprise a mirror layer made of an electrically conductive material. In yet another embodiment of the present invention, said first reflecting/mirror layer is made of an electrically conductive metal or an electrically conductive metal film. In a further preferred embodiment, said minor layer is made of gold or a gold film. In a preferred embodiment of the present invention, the reflecting/mirror layer made of gold has a thickness of about 10 to about 30 nm. In an also preferred embodiment, said mirror layer is made of titan or a titan film. In a preferred embodiment of the present invention, the reflecting/mirror layer made of titan has a thickness of about 10 to about 70 or about 10 to about 60 nm.

The fourth semi-reflecting layer of devices according to the second aspect of the invention may be made from the same material or a different material as the first reflecting layer. Typically, the fourth semi-reflecting layer will have a thickness in the range of 1 to 100 nm. If the fourth semi-reflecting layer is made from the same material as the first reflecting layer, it will be thinner than the first reflecting layer. In such cases, the fourth reflecting layer will have about 5% to about 70%, preferably about 5 to about 60%, more preferably about 5% to about 50% such as about 5%, about 10%, about 15%, about 20%, about 25%, about 30%, about 35%, about 40%, about 45% or about 50% of the thickness of the first reflecting layer. The thinner the fourth semi-reflective layer is compared to the first reflecting layer, the less reflection will occur at the fourth semi-reflecting layer. In a preferred embodiment, the fourth semi-reflecting layer will have a thickness of about 1 nm to about 40 nm, preferably of e.g. about 2 nm to about 35 nm or about 5 nm to about 30 nm such as about 10 nm, about 15 nm, about 20 nm or about 25 nm.

The fourth semi-reflecting layer may be continuous or discontinuous. If the fourth semi-reflecting layer is discontinuous, it may be a nanoparticle layer which preferably comprises island-like structures having a size of about 5 nm to about 50 nm.

Furthermore, in the first and second aspect of the present invention, the polymer layer comprises a biodegradable polymer and/or a polymer capable of swelling/shrinking and/or a polymer capable of absorbing biomolecules.

In a preferred embodiment of the first and second aspect of the present invention, the polymer layer is a biodegradable polymer layer degradable by biomolecules comprising enzymes and/or catabolic metabolites. In yet another preferred embodiment of the first and second aspect of the invention, the polymer layer is a polymer capable of swelling/shrinking upon contacting biomolecules comprising ionic molecules. In an also preferred embodiment of the first and second aspect of the invention, the polymer layer is a polymer layer capable of absorbing biomolecules comprising enzymes and/or catabolic metabolites.

In a preferred embodiment of the first and second aspect of the present invention, said biodegradable polymer layer is selected from the group of polymers comprising PLA, PLGA, PHB, and polyvinylcaprolactame. In another preferred embodiment of the first and second aspect, said polymer layer capable of swelling/shrinking is selected from the group of polymers comprising polyacrylic acid derivates and polyvinylpyrrolidone derivatives. In another embodiment of the first and second aspect of the invention, the polymer layer capable of absorbing biomolecules is selected from the group of polymers comprising vinyl polymers having various side-chain groups and polycondensation products such as polyesters, polyamides, polyimides, polyurethanes and polyureas.

In a further preferred embodiment of the first and second aspect of the present invention, the third polymer layer has a thickness of about 5 nm to about 1000 nm prior to being contacted by said biomolecules. In another preferred embodiment of the first and second aspect of the present invention, the polymer layer has a thickness of about 100 nm to about 500 nm prior to being contacted by said biomolecules. In particularly preferred embodiments of the second aspect of the invention, the third polymer layer will have a thickness of about 2 nm to about 200 nm, of about 3 nm to about 150 nm, of about 5 nm to about 145 nm such as about 10 nm, about 20 nm, about 30 nm, about 40 nm, about 50 nm, about 60 nm, about 70 nm, about 80 nm, about 90 nm, about 100 nm, about 110 nm, about 120 nm, about 130 nm or about 140 nm.

The second transparent layer of the second aspect of the invention will typically be made from materials giving a higher refractive index than the materials used for the first reflecting layer, the fourth semi-reflecting layer and the third polymer layer. Such materials will typically be selected from the group comprising TiO2, SiN, Ta₂O₅, ZnS, CeO2, Nb₂O₅, ZrO₂, ZrO₂⁺, TiO₂, TiO or Ti₃O₅. The transparent layer will typically have a thickness of about 5 nm to about 150 nm, of about 10 to about 140 nm such as about 15 nm, about 20 nm, about 30 nm, about 40 nm, about 50 nm, about 60 nm, about 70 nm, about 80 nm, about 90 nm, about 100 nm, about 110 nm, about 120 nm or about 130 nm.

In the afore-mentioned embodiments of the second aspect of the invention, the refractive index n2 will be >n1, n3 and n4. Preferably n1 and n4 will moreover be <n3.

Typically, n2 may be in the range of about 1 to about 3, such as about 1.9, about 2, about 2.1, about 2.2, about 2.3, about 2.4, about 2.5, about 2.6, about 2.7, about 2.8 or about 2.9.

Typically, n1 and/or n4 will be in the range of about 0.1 to about 2.0 such as about 0.2, 0.3, 0.4, 0.5, 0.6, 0.7, 0.8, 0.9, 1.1, 1.2, 1.3, 1.4, 1.5, 1.6, 1.7, 1.8 or 1.9.

Typically, n3 will be in the range of about 0.5 to about 2.5 such as about, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3 or about 2.4.

The refractive index n2 may be > than the refractive index n1, n3 and n4 by at least about 10%, preferably by at least about 15%, at least about 20%, at least about 25%, at least about 30%, at least about 35%, at least about 40%, at least about 45% or at least about 50%.

In still another embodiment of the second aspect of the present invention, the device described above comprises an additional transparent layer above the fourth semi-reflecting layer made from the same material as the second transparent layer.

In still another embodiment of the first and second aspect of the present invention, the device described above comprises an additional carrier layer on the reflecting layer(s) being positioned on the opposite of said polymer layer.

In yet a further aspect of the first and second aspect of the present invention, the device also comprises a reference device.

The present invention also provides a method for preparing a device of the first aspect of the invention. This method comprises at least the steps of
(a) providing a first reflecting layer
(b) applying a polymer layer onto said first reflecting layer
(c) applying a second reflecting layer onto said polymer.

An additional step may be added to the method for preparing a device described above. Thus, the method may comprise the additional step of applying an additional layer with a high refractive index onto said first reflecting layer before the polymer layer is applied onto said additional layer.

The method may also comprise the additional step of providing a carrier and applying said first reflecting layer onto said carrier.

The present invention also provides a method for preparing a device of the second aspect of the invention. This method comprises at least the steps of
a. Providing a first reflecting layer having a refractive n1;
b. Applying a second transparent above said first reflecting layer having a refractive n2;
c. Applying a third polymer layer above said second transparent layer having a refractive n3;
d. Applying a fourth semi-reflecting layer above said third polymer layer having a refractive n4,
wherein n2 is >n1, n3 and n4.

This method may also comprise the additional step of providing a carrier and applying said first reflecting layer onto said carrier.

In yet another aspect of the methods of the present invention, the polymer layer is applied by dip coating or film printing.

In the methods of the present invention relating to devices of the first and second aspect of the invention, the reflecting layers and/or semi-reflecting layers are applied by sputter-coating, by evaporation or by chemical reactions, such as direct application of gold through a chemical reaction using the reduction of HAuCl₄. Also, the additional layer with high refractive index and/or the transparent layer may be applied by sputter-coating, by evaporation or by chemical reactions.

The present invention relates in one aspect to a method for analyzing the age and/or quality of a natural product comprising foods and cosmetic products. This method comprises the following steps:
(a) providing a device as described above
(b) contacting said device with a natural product
(c) determining the colour of said device
(d) comparing the colour of said device to the colour of a reference device
(e) determining the age and/or quality of said natural product according to this comparison.

The afore described method for analyzing the age and/or quality of a natural product comprises in a further embodiment a step (step b) listed above) wherein a reflecting layer of said device is being contacted in step b) with said natural product in such a way that biomolecules are allowed to penetrate this reflecting layer and contact the polymer layer.

In a preferred embodiment, a device as described above is used for the analysis of the age and/or quality of a natural product comprising foods and cosmetical products.

The present invention relates in a further preferred embodiment to the use of a device as described above for the analysis of the age and/or quality of a natural product by detecting microorganisms present in the natural product.

The present invention relates in a further preferred embodiment to the use of a device as described above for the analysis of the age and/or quality of a natural product by detecting enzymes and/or catabolic metabolites of microorganisms and/or of the natural product via the degradation of said biodegradable polymer by said enzymes and/or catabolic metabolites and/or via swelling/shrinking of said polymer layer upon contacting said biomolecules and/or absorption of biomolecules by and into said polymer layer.

### DESCRIPTION OF THE FIGURES

FIG. 1A:

Exemplary use of a device according to the invention to analyze the age and/or quality of meat. In the left case, both, the device according to the invention (which may also be a combination of different devices as set out in the detailed description of the invention) and the reference device, are shown. The device according to the invention is formed as stripe in the left picture and as square in the right picture. The reference device comprises three colours indicating the quality of the product ranging from “ok” to “harmful”. The patterning of the device depicted here does not show a certain embodiment, it is rather meant to illustrate that the device may have different colours, e.g. green, yellow and red.

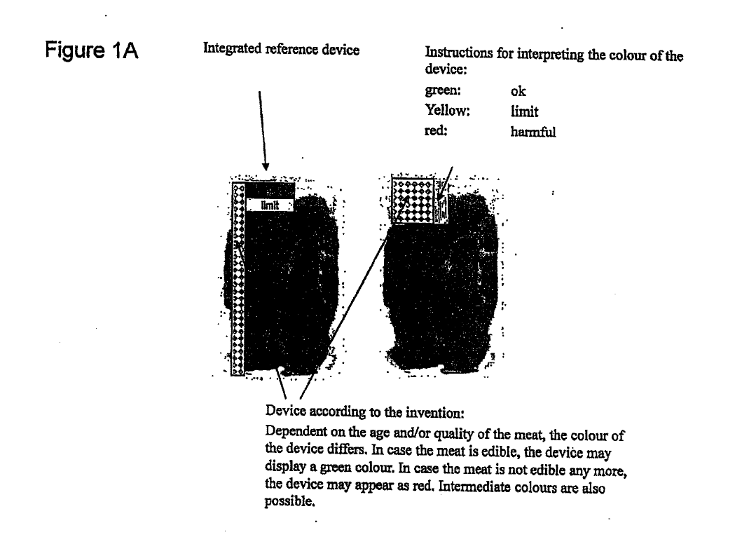

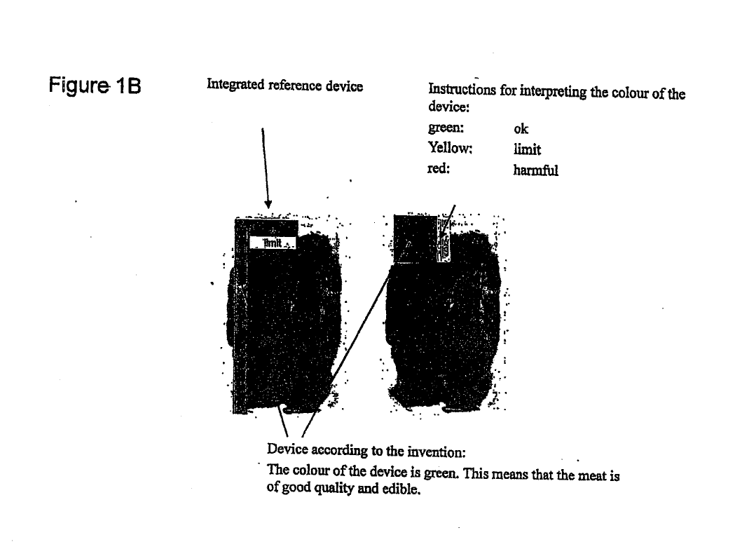

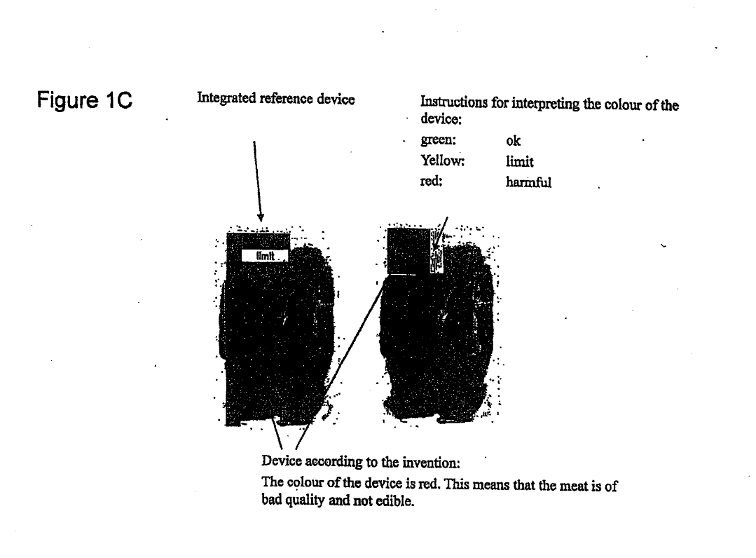

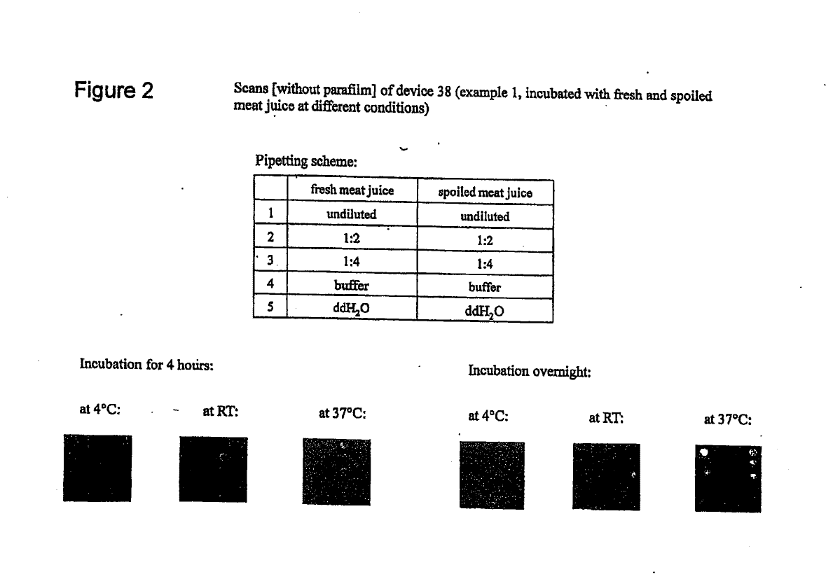

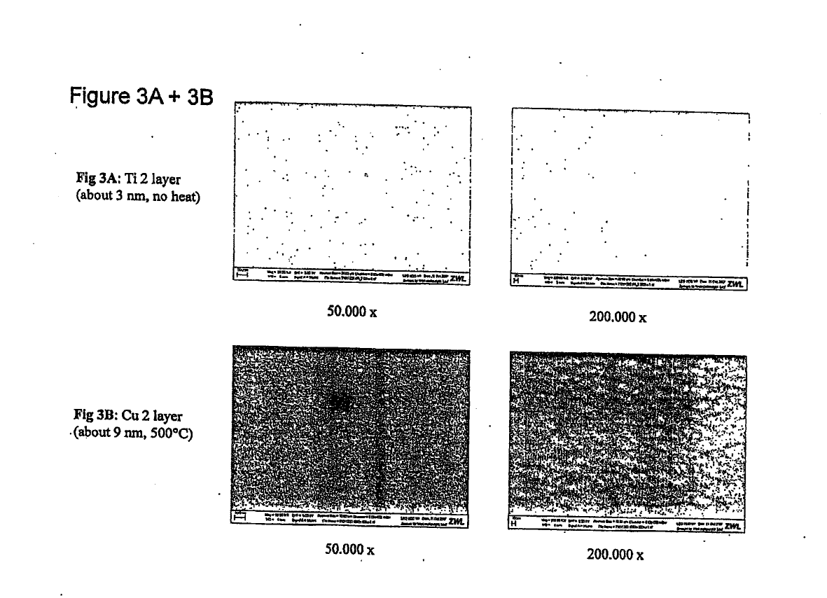

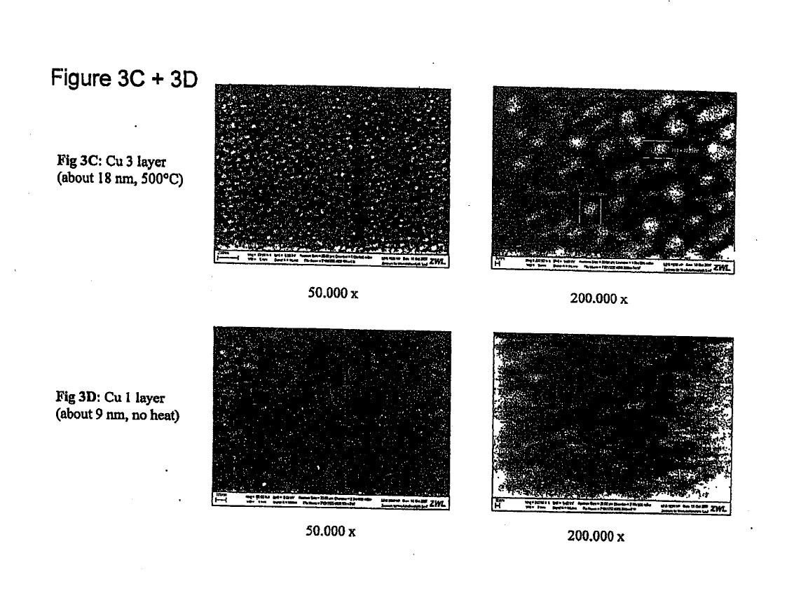

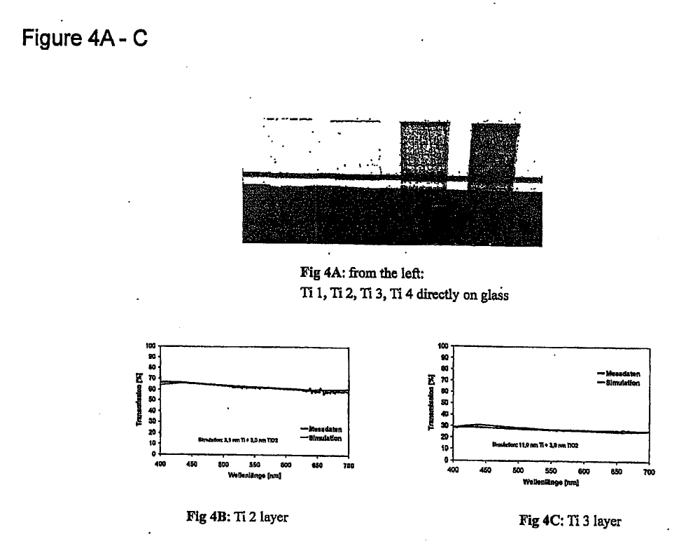

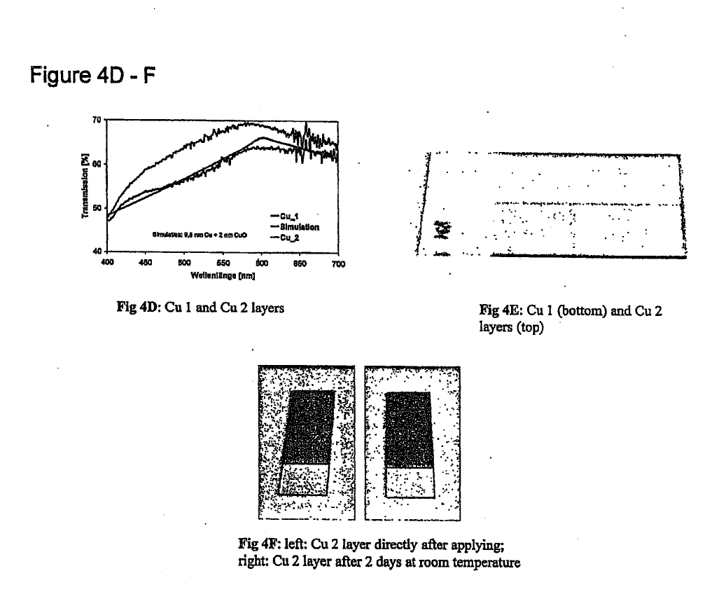

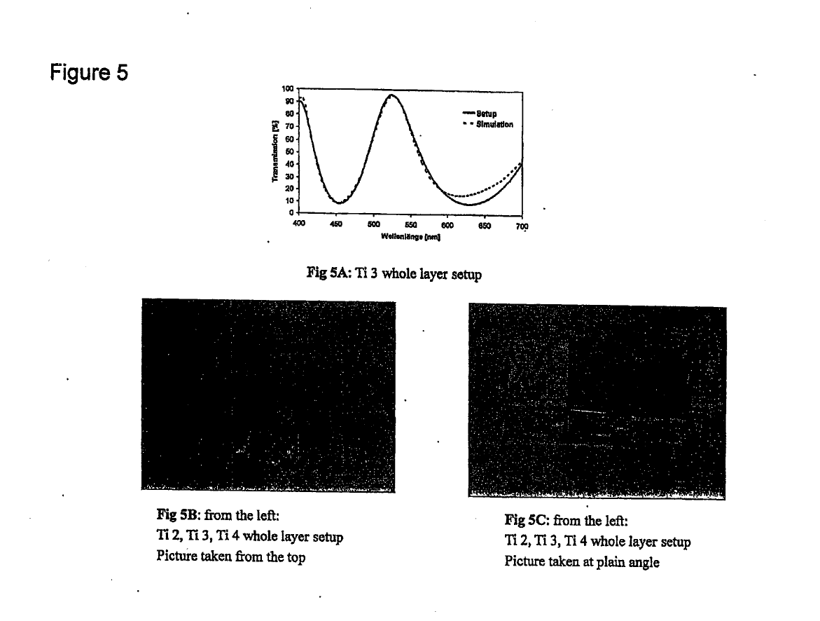

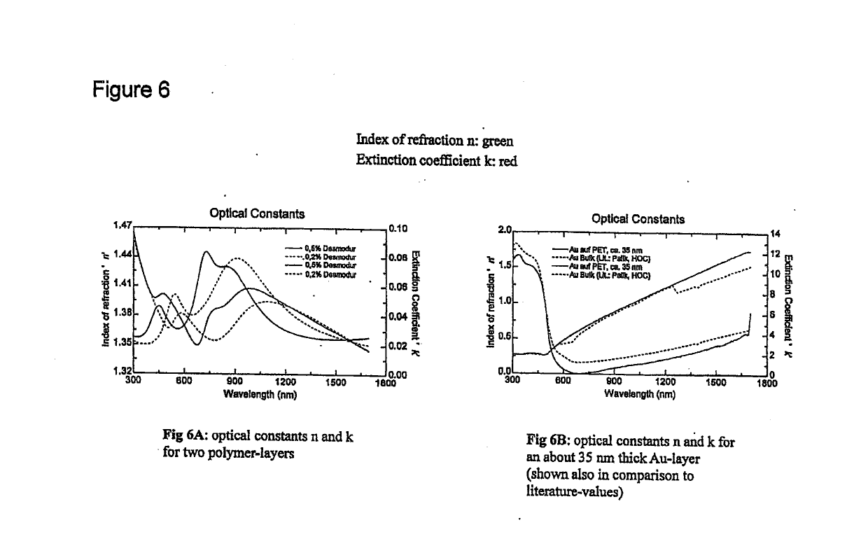

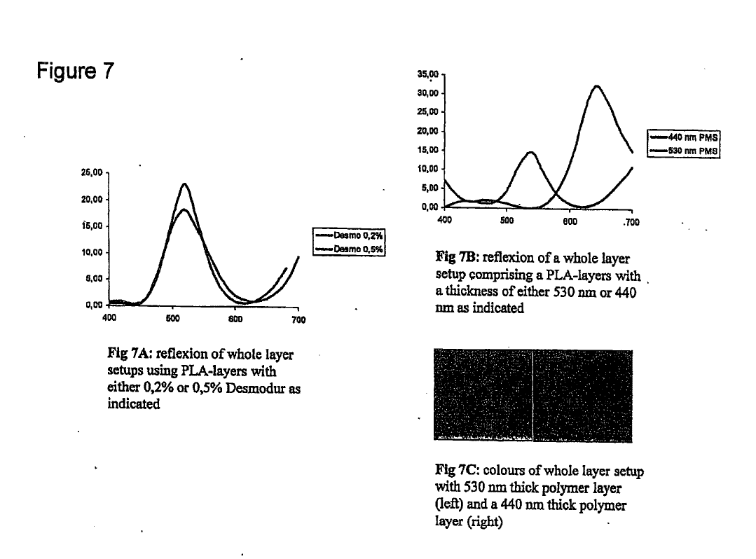

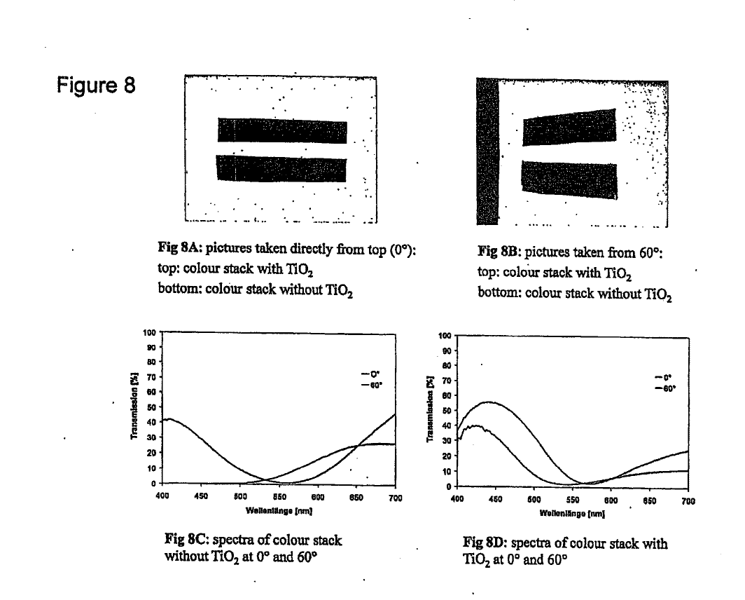

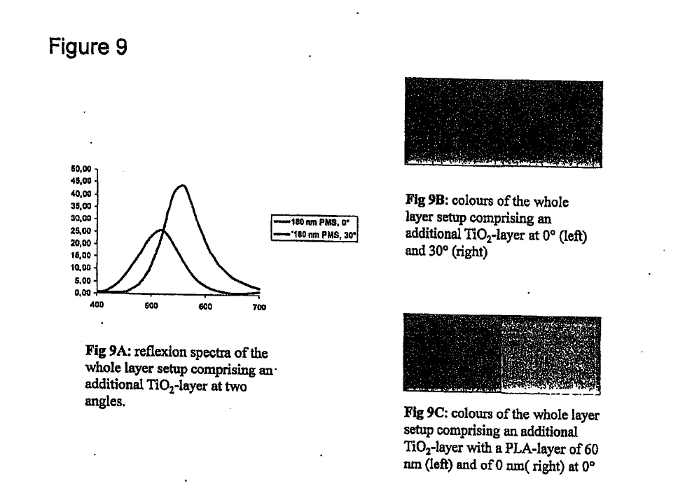

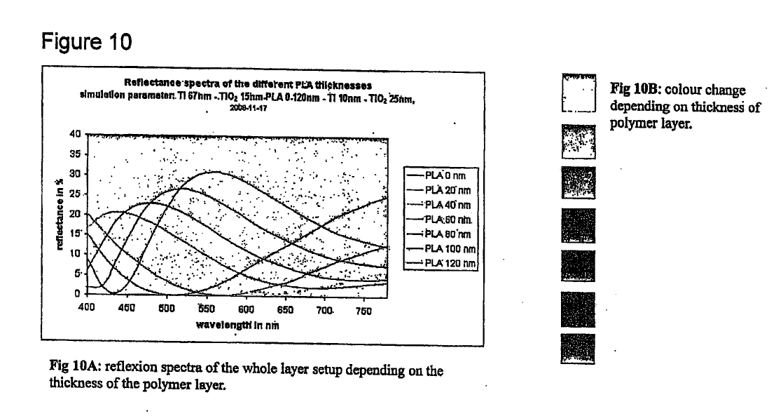

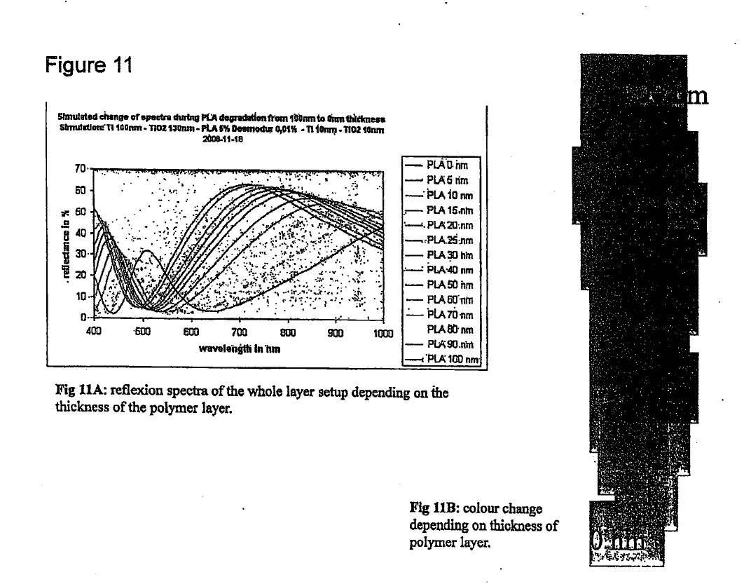

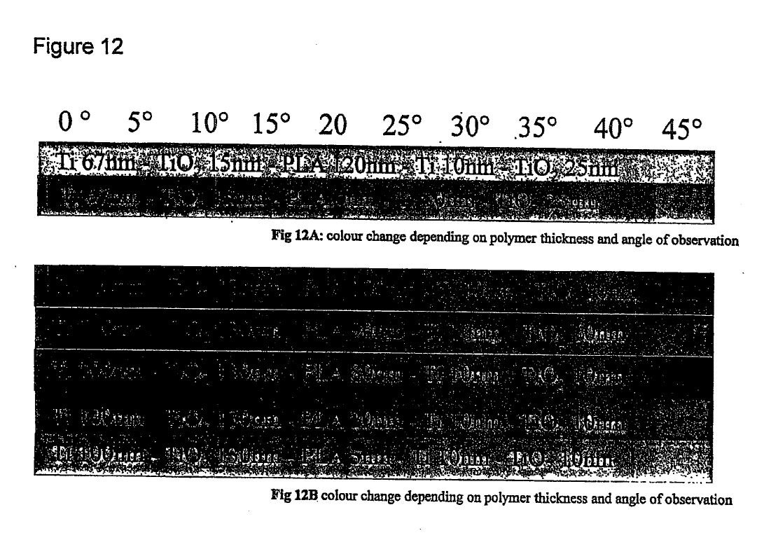

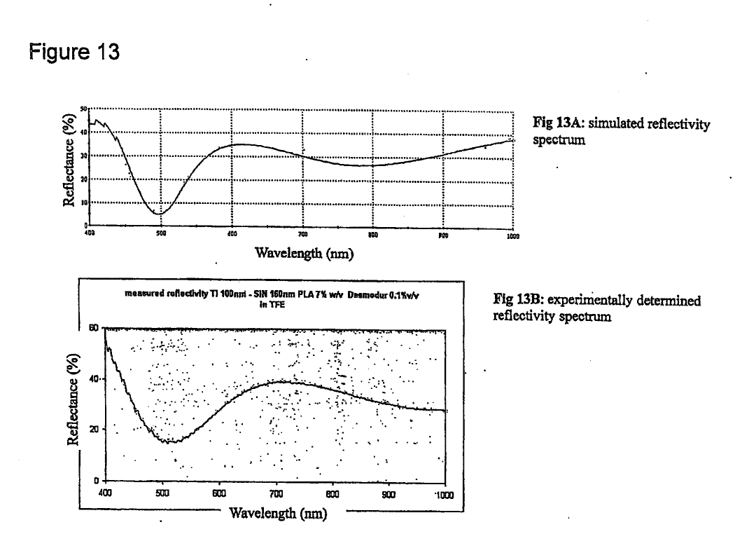

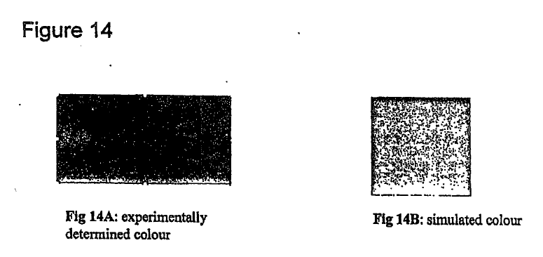

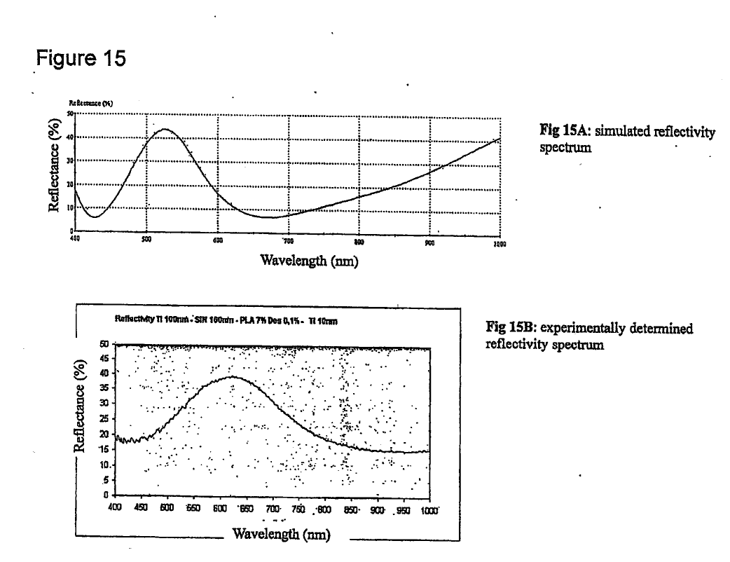

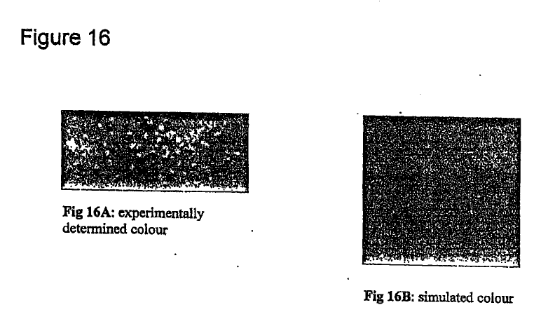

FIG. 1B:

Exemplary readout of the device according to the invention in case of meat of good quality. The device according to the invention either formed as a stripe or as a square is green signaling in combination with the reference device or the instructions that the meat is of good quality and edible.

FIG. 1C:

Exemplary readout of the device according to the invention in case of old meat or meat of bad quality. The device according to the invention either formed as a stripe or as a square is red signaling in combination with the reference device or the instructions that the meat is of bad quality and not edible any more.

FIG. 2:

Scans of a device according to the invention for the analysis of fresh and spoiled meat juice. A device comprising an Inconnel-, a 10% PLA, 10⁻⁴% Desmodur polymer- and an Au-layer (sputter coated for 4×30 sec) was incubated with meat juice as indicated at different temperatures and for different times (see example 2 for details).

FIG. 3:

Electron microscopy pictures of different titan- and copper-layers. The layers (see example 3 for thicknesses and sputter coating conditions of Ti 2 [FIG. 3 A], Cu 2 [FIG. 3B], Cu 3 [FIG. 3C], Cu 1 [FIG. 3D]) were sputter coated directly on glass slides and pictures of the surfaces were taken at the indicated magnifications.

FIG. 4:

Pictures of different layers (FIGS. 4A, E and F) as well as experimental results for transmission spectra compared to simulations (FIG. 4B, C, D). The semi-transparent titan layers of different thicknesses (Ti 1 about 1 nm, Ti2 about 3 nm, Ti 3 about 11 nm, Ti 4 about 14 nm) shown in FIG. 4A were all directly sputter coated onto glass slides and the transmission spectra were determined for Ti 2 and Ti 3 and compared to simulations (see FIGS. 4B and 4C). FIG. 4D shows such a comparison for Cu 1 and Cu 2, whereas FIG. 4E depicts Cu 1 and Cu 2 in a visual, direct comparison of pictures taken. FIG. 4F shows the colours of Cu 2 layers depending on the incubation times, namely either almost no incubation directly after applying the layer and after two days of incubation at room temperature.

FIG. 5:

Experimental results for the transmission spectrum of the whole layer setup Ti 3 compared to a simulation (FIG. 5A) and pictures of the whole layer setups Ti 2, Ti 3, Ti 4. In contrast to the graphs depicted in FIG. 4, now a whole setup of layers (Ti 3 setup) is shown compared to a simulation of such a whole layer setup. The brilliant colours of the whole layer setups Ti 2, Ti 3 and Ti 4 are shown in FIGS. 5B and C from two different angles. For layers of whole layer setups see example 3.

FIG. 6:

Determination of optical constants n and k for two different polymer layers and a gold-layer. The index of refraction n as well as the extinction coefficient k were determined for two polymer layers; either 10% PLA with 0.5% Desmodur or 10% PLA with 0.2% Desmodur (see FIG. 6A as indicated). Furthermore, both constants were also determined for an about 35 nm thick Au-layer and compared to literature-values (depicted in FIG. 6B as indicated).

FIG. 7:

Simulations of the reflexion spectra of different whole layer setups and of their colours. The experimentally determined optical constants were used to simulate the reflexion spectra of two identical setups differing only in the concentration of Desmodur used in the polymer layer (FIG. 7A). FIG. 7B shows the corresponding spectra for two whole layer setups differing only in the thickness of the polymer layer; in blue, the spectrum for a setup with a 530 nm thick polymer layer is shown; this corresponds to a green colour as depicted in FIG. 7C, left. In red, the spectrum for a setup with a 440 nm thick polymer layer is shown; this corresponds to a red colour as depicted in FIG. 7D, right.

FIG. 8:

Effect of an additional layer with a high refractive index on the colour of an interference system. FIGS. 8A and 8B show two different colour stacks from two different angles: in FIG. 8A, the picture is taken directly from the top, whereas in FIG. 8B the picture is taken from a 60° angle. The colour stack shown in the top of these pictures comprises an additional TiO₂-layer whereas the colour stack in the bottom lacks such a layer. FIGS. 8C and 8D show the reflexion spectra of said two different colour stacks at two angles as indicated.

FIG. 9:

Simulations of the reflexion spectra of a whole layer setup comprising an additional layer and of their colours. The experimentally determined optical constants were used to simulate the reflexion spectra at two different angles as indicated for the following setup:

Continuous Au-layer—100 nm TiO₂-layer—180 nm 10% PLA, 0.5% Desmodur—10 nm Ti (+ an additional native 25 nm TiO₂-layer), shown in FIG. 9A. FIG. 9B shows the corresponding colours of such a device at 0° and 30°. The effect of a decreasing polymer layer on the colour is depicted in FIG. 9C, wherein the layer has a thickness of either 60 nm or is not present at all (right).

FIG. 10:

Simulations of the reflexion spectra of a whole layer setup depending on the thickness of the polymer layer. FIG. 10A shows how the reflection spectra shift depending on the colour of polymer layer. FIG. 10B shows how the colour changes depending on the thickness of the polymer layer. The set-up is detailed in Example 7 as set up 1.

FIG. 11:

Simulations of the reflexion spectra of a whole layer setup depending on the thickness of the polymer layer. FIG. 11A shows how the reflection spectra shift depending on the colour of polymer layer. FIG. 11B shows how the colour changes depending on the thickness of the polymer layer. The set-up is detailed in Example 7 as set up 1.

FIG. 12:

Simulations of the reflexion spectra of a whole layer setup depending on the thickness of the polymer layer and the observation angle. FIG. 12A shows how colours change depending on the colour of polymer layer and the angle of observation for set up 1 of Example 7. FIG. 12B shows how colours change depending on the colour of polymer layer and the angle of observation for set up 2 of Example 7.

FIG. 13

Comparison of simulated and experimentally determined reflectivity spectra of the set up of example 9. FIG. 13A shows the simulated reflection spectrum while FIG. 13B shows the measured reflection spectrum of the set up.

FIG. 14:

Comparison of simulated and experimentally determined colours of the set up of example 9. FIGS. 14A and B show the colours as determined for the simulated (B) and the experimentally tested (A) set up.

FIG. 15

Comparison of simulated and experimentally determined reflectivity spectra of the set up of example 10. FIG. 15A shows the simulated reflection spectrum while FIG. 15B shows the measured reflection spectrum of the set up.

FIG. 16:

Comparison of simulated and experimentally determined colours of the set up of example 10. FIGS. 16A and B show the colours as determined for the simulated (B) and the experimentally tested (A) set up.

### DETAILED DESCRIPTION OF THE INVENTION

As has been set out above, there is a need for a device which allows the analysis of the age and/or quality of a natural product by the consumer.

The present invention provides devices and methods for solving this need. While describing in detail exemplary embodiments of the present invention, definitions important for understanding the present invention are also given.

The present invention will be described with respect to particular embodiments and with reference to certain drawings and figures but the invention is not limited thereto but only by the claims. The drawings and figures as described are only schematic and non-limiting. In the drawings and figures, the size of some of the elements may be exaggerated and not drawn on scale for illustrative purposes

As used in this specification and in the appended claims, the singular forms of “a” and “an” also include the respective plurals unless the context clearly dictates otherwise.

In the context of the present invention, the terms “about” and “approximately” denote an interval of accuracy that a person skilled in the art will understand to still ensure the technical effect of the feature in question. The term typically indicates a deviation from the indicated numerical value of ±10% and preferably ±5%.

It is to be understood that the term “comprising” is not limiting. For the purposes of the present invention the term “consisting of” is considered to be a preferred embodiment of the term “comprising of”. If hereinafter a group is defined to comprise at least a certain number of embodiments, this is meant to also encompass a group which preferably consists of these embodiments only.

The term “natural product” in the context of the present invention comprises any product which is subject to spoilage and/or decay and, therefore, possesses a certain time frame in which it may be used according to its purpose. As set out in the background section, using an e.g. spoiled natural product such as eating spoiled food may lead to major health problems. Foods form a very large class of such natural products. This class is comprised of fish, meat, milk products, vegetables, carbohydrate-containing foods such as bread, and the like. Another class of natural products comprises cosmetic products, which are also subject to spoilage and/or decay, e.g. in case of inadequate storage and/or delivery conditions and aging processes. The term “natural product” in the context of this invention does not define a “natural product” in a way that it has to be untreated. The natural product may be treated or untreated. Any natural product according to the invention may be treated and/or e.g. used in preparation processes, such as e.g. cooking, baking, boiling, freezing, and the like. “Natural” in the context of the invention rather implies that the product, although it may be pretreated, is still a substrate for spoilage and/or decay processes by e.g. microorganisms. The natural product may also be packaged in any way known to the person skilled in the art.

The term “age and/or quality” relates to the natural product as defined above. As mentioned before, natural products possess different time frames, in which they may be used according to their purposes. A very important aspect is the age of the product because of the correlation of contamination by e.g. microorganisms and time. The longer the incubation time, the more concentrated and severe the contamination may be. In case of inadequate storage and/or delivery conditions, this correlation may change and favour an even more severe contamination within a shorter time. For this reason, the natural product should always be subjected to controls even if it is not incubated for a long time. Therefore, “age” and “quality” in the context of the present invention mainly refer e.g. in case of foods to the edibility of such foods.

Therefore, a device according to the invention is used in a preferred embodiment to analyze if the cold chain of foods has been handled correctly. As set out above, most of the microorganisms responsible for the spoilage of foods preferably proliferate at 37° C. Therefore, many foods, e.g. meat, are stored and transported at temperatures below 37° C., preferably at 4° C. or even frozen at −20° C. to maintain an unfavourable temperature range for such microorganisms. As transport includes different storage areas maintained at such low temperatures as e.g. cold storage houses or adequate transport vehicles, the whole delivery process from the place of production to the place of offering such foods (e.g. a supermarket) is referred to as the cold chain. Therefore, the device according to the invention may be used by the consumer of the natural product to analyze if the natural product indeed has been handled according to the cold chain. Alternatively, the person involved in presenting and selling the product (e.g. an employee of a supermarket) may check at the arrival the quality of the natural product to decide whether storage and/or transport have been handled according to the cold chain and, therefore, whether the natural product may be presented to the consumer.

The term “biomolecules” in the context of the present invention defines molecules present in or secreted from the natural product to be analysed and/or present in or secreted from any further object associated with said natural product, e.g. a microorganism. Therefore, the biomolecules may derive from e.g. a food such as meat or from a microorganism associated with said meat. Preferably, such biomolecules comprise enzymes, such as phospholipases, hydrolases, pronases, proteinases (such as proteinase K), esterases of different types, lipases and the like. Biomolecules according to the present invention also comprise any molecules present in or secreted from the natural product to be analysed and/or present in or secreted from any further object associated with said natural product, e.g. a microorganism, of non-enzymatic origin. Some of these molecules may be capable of degrading the biodegradable polymer layer, i.e. for example intermediates of the metabolism of such microorganisms associated with said natural product. Such non-enzymatic molecules are defined in the context of the invention as catabolic metabolites. Examples for such catabolic metabolites comprise volatile acids, volatile bases, volatile aldehydes, volatile mercaptans and sulfur compounds. In preferred embodiments, enzymes and/or catabolic metabolites are able to degrade the polymer layer of the devices described herein. However, in further preferred embodiments of the invention, such molecules (enzymes as well as catabolic metabolites) may not degrade the polymer layer of the device, but are rather absorbed by and into the polymer layer. Yet another class of biomolecules comprises molecules secreted from or released from the natural product and/or any further object associated with said natural product, e.g. a microorganism, which are products of enzymatic reactions or of non-enzymatic reactions and lead to a change in ionic strength and, therefore, ultimately to a change in the pH. Examples for such molecules as products of enzymatic reactions are glucose, urea and organic esters which are present as gluconic acid, NH₃ and free organic acids following the respective enzymatic reactions. The latter mentioned molecules may lead to a change in the pH in case they are secreted from a natural product to be analysed and/or any further object associated with said natural product. In some cases, a at least partial mechanical destruction of the natural product or of the packaging of the natural product may lead to the release of said molecules affecting the pH in the environment of the natural product to be analyzed.

The present invention describes a device according to a first aspect of the invention comprising two reflecting layers. Combined with a polymer layer in between those two layers, the device can be regarded as optical sensor on the basis of a Fabry-Perot filter as described below in more detail. At first, the nature of the “reflecting layer” itself is described, followed by a description of the “polymer layer” and how these layers can be combined as to result in the setup of a Fabry-Perot filter.

The term “reflecting layer” (as used in the context of devices corresponding to the first and second aspect of the invention as mentioned above) describes a layer which is reflecting incident light. In the setup used herein, the most preferred light source is daylight or any other light source resembling daylight, e.g. light bulbs, neon light and so on. The reflecting layer may also be called “mirror layer”. In a preferred embodiment, this layer may be an electrically conductive material. In a further preferred embodiment, the layer may be an electrically conductive metal or metal film, such as preferably gold (or a gold film) or even more preferably titan (or a titan film). It may also be any other metal or an alloy such as CrNi. In another embodiment, aluminium or Inconnel is used as mirror layer. It is in a further preferred embodiment also possible to use TiAl (titanaluminide) as mirror layer. TiAl is highly reflective and may have the advantage of being more stable than aluminium. In case titan is used as the reflecting layer, the person skilled in the art knows that a TiO₂-layer will naturally always form on an exposed titan surface. Thus, in case titan is used as reflecting layer with an exposed surface, a TiO₂-layer with a thickness of about 10 nm to about 30 nm will also be present as ultimate surface layer and is meant to be also comprised when using titan. The person skilled in the art knows that the thickness of the spontaneously formed TiO₂ layer will depend on the thickness of the Ti layer. Typically the spontaneously formed TiO₂ layer will be 50% of the thickness of the Ti layer. Preferably, the material used for the reflecting layer is inert to any reactions with the natural product. The above explanations explicitly apply to the first reflecting layer of devices according to the second aspect of the invention.

The nature of the reflecting layer (as used in the context of devices corresponding to the first and second aspect of the invention as mentioned above) can be described as continuous. The term “continuous reflecting layer” in the context of the present invention means that the reflecting layer does not comprise any island-like isolated structures or a plurality of discrete islands or a layer of islands or metallic island films wherein the islands are structured in a more or less regular arrangement. It needs to be understood, however, that due to the methods of preparing such devices or due to the material used as reflecting layer small holes, openings or gaps may be present in said reflecting layer. However, such holes, openings or gaps are never present in a structured, regular arrangement and do not reflect any desired structures. In no case, however, the term “continuous” means that the reflecting layer is not permeable to biomolecules as defined above. The permeability of the reflecting layer mainly depends on the thickness of the layer and its composition/material, as set out below. As set out in the paragraph above, the reflecting layer may be made of an electrically conductive material. Therefore, in a further preferred embodiment, the continuous reflecting layer as described above is also conductive. This leads to a “continuous and conductive reflecting layer”, which is preferably used as mirror layer according to the invention. Furthermore, the reflecting layer should be stabile in biological buffers and have a very smooth surface. The reflecting layer may be about 0.5 nm to about 500 nm thick, preferably it may be about 1 nm to about 100 nm thick, more preferably it may be about 5 nm to about 50 nm thick and even more preferably it may be about 10 to about 30 nm thick. In certain embodiments, it may be about 10 nm or 60 nm thick. The above explanations explicitly apply to the first reflecting layer of devices according to the second aspect of the invention.

As mentioned above, two reflecting layers are used in the setup of devices according to the first aspect of the present invention. The two layers may in one embodiment of the invention be identical. However, in other embodiments of the invention, the two layers may be different, mainly with regard to their thickness (thus, one layer may be about 60 nm thick, whereas the other layer may be about 10 nm thick) and/or material/composition. In case the two reflecting layers are identical, there is no need to differentiate between the two reflecting layers; either one of them can be exposed on one side to the natural product to be analyzed, whereas on the other side the polymer layer is posited. In case the two layers differ from each other, it is intended that one specific reflecting layer is exposed to the natural product. The thicker this layer, the less biomolecules penetrate the layer to the other side and the longer it takes the biomolecules to contact the layer on the other side of the reflecting layer. Therefore, it is possible to adjust the sensitivity of the whole device by adjusting the thickness of the reflecting layer which is directly exposed to the natural product. Also, as already mentioned above, the material/composition of this reflecting layer may be adjusted in order to ultimately achieve a desired response at a specific sensitivity within a certain time frame. In one preferred embodiment of the invention, the device should, for example, respond to a biomolecule secreted from the natural product by signalling within a short period of time; to achieve this, the reflecting layer exposed to the natural product may be rather thin and/or may be composed of a material/composition which can be more easily and, therefore, also faster penetrated by said biomolecules. The above explanations explicitly apply to the first reflecting layer of devices according to the second aspect of the invention.

The fourth semi-reflecting layer of devices according to the second aspect of the invention may be made from the same material or a different material as the first reflecting layer. Typically, the fourth semi-reflecting layer will have a thickness in the range of 1 to 100 nm. If the fourth semi-reflecting layer is made from the same material as the first reflecting layer, it will be thinner than the first reflecting layer. In such cases, the fourth reflecting layer will have about 5% to about 70%, preferably about 5 to about 60%, more preferably about 5% to about 50% such as about 5%, about 10%, about 15%, about 20%, about 25%, about 30%, about 35%, about 40%, about 45% or about 50% of the thickness of the first reflecting layer. The thinner the fourth semi-reflective layer is compared to the first reflecting layer, the less reflection will occur at the fourth semi-reflecting layer. In a preferred embodiment, the fourth semi-reflecting layer will have a thickness of about 1 nm to about 40 nm, preferably of e.g. about 2 nm to about 35 nm or about 5 nm to about 30 nm such as about 10 nm, about 15 nm, about 20 nm or about 25 nm.

The fourth semi-reflecting layer may be continuous or discontinuous. If the fourth semi-reflecting layer is continuous, the above explanations given for the reflecting layers equally apply.

If the fourth semi-reflecting layer is discontinuous, it may be a nanoparticle layer which preferably comprises island-like structures having a size of about 5 nm to about 50 nm.

In the context of the invention, the term “nanoparticle layer” describes a layer made of a plurality of nanostructures.

These nanostructures may be positioned on the polymer layer as structures that are isolated from each other. These isolated structures may take various geometric forms such as squares, circles etc. The structures may, of course, also have an irregular shape. Given that the structures irrespective of their shape are isolated from each other, they may be designated as “island-like” nanostructures or “island-like structures”.

In another embodiment the nanostructure layer is made from a meshwork of nanostructures, i.e. nanostructures which are connected with each other. Such structures may have a regular shape, i.e. take the form of a grid or an irregular shape, i.e. an irregular meshwork. The knots of such meshworks may take the shape of the above-described “island-like” structures.

The nanoparticle layer may also be a combination of these two embodiments, i.e. comprise partly isolated nanostructures and a meshwork of nanostructures.

The nanoparticle layer is typically translucent for incident light and for light reflected by the mirror layer.

Therefore, in preferred embodiments of the invention, the diameter of the island-like structures and/or of the knots of the meshwork of nanoparticles is smaller than the wavelength of the incident and reflected light.

In a preferred embodiment, the diameter of the island-like structures and/or the knots of the meshwork of nanostructures are in the range of about 1 nm to about 100 nm, preferably about 5 nm to 50 nm and more preferably of about 2 to about 20 nm.

In a preferred embodiment of the invention, the island-like structure and/or the meshwork of the nanoparticle layer are made of a metal. In a further preferred embodiment, this metal is gold, titanium or copper. As for the reflecting layer, the material for the nanoparticle layer is in a preferred embodiment inert to any reaction with the natural product. Furthermore, also the nanoparticle layer is configured such that biomolecules defined above are able to penetrate said layer in order to contact the layer on the other side of the reflecting layer.

The nanoparticle layer may have the thickness indicated above for the fourth semi-reflecting layer.

It is possible to adjust the sensitivity of the whole device by adjusting the thickness of the semi-reflecting layer which may be directly exposed to the natural product.

Also, as already mentioned above, the material/composition of the reflecting and also the semi-reflecting layer may be adjusted in order to ultimately achieve a desired response at a specific sensitivity within a certain time frame. In one preferred embodiment of the invention, the device should, for example, respond to a biomolecule secreted from the natural product by signalling within a short period of time; to achieve this, the semi-reflecting layer exposed to the natural product may be rather thin and/or may be composed of a material/composition which can be more easily and, therefore, also faster penetrated by said biomolecules.

The second transparent layer of the second aspect of the invention will typically be made from materials giving a higher refractive index than the materials used for the first reflecting layer, the fourth semi-reflecting layer and the third polymer layer. Such materials will typically be selected from the group comprising TiO2, SiN, Ta₂O₅, ZnS, CeO2, Nb₂O₅, ZrO₂, ZrO₂⁺, TiO₂, TiO or Ti₃O₅. One advantage of using a transparent layer having a higher refractive index than the other layers is that the colour of the device will not substantially change when looking at the device at different angles such as 90° or 45°. Given that the colours of the device will not depend substantially on the angle of observation, this ensures that an observed colour change is indeed the result of e.g. meat enzymes degrading the polymer layer.

In still another embodiment of the second aspect of the present invention, the device described above comprises an additional transparent layer above the fourth semi-reflecting layer made from the same material as the second transparent layer. Such a second transparent layer which may be designated as fifth transparent layer may further insure that the colours of the device do not substantially depend on the angle of observation. It will have a refractive index n5 which is again >n1, n3 and n4 but ≦n2.

The transparent layer (be it the second or fifth transparent layer) will typically have a thickness of about 1 to about 500 m, of about 5 nm to about 150 nm, of about 10 to about 140 nm such as about 15 nm, about 20 nm, about 30 nm, about 40 nm, about 50 nm, about 60 nm, about 70 nm, about 80 nm, about 90 nm, about 100 nm, about 110 nm, about 120 nm or about 130 nm.

The person skilled in the art will furthermore be aware that optimisation experiments may be necessary with respect to the thickness and the materials of the first through fourth layer.

Thus for certain set-ups of devices in accordance with the second aspect of the invention, one may use a first reflecting layer of Ti with a thickness of 50 to 70 nm, a second transparent layer of TiO₂ with a thickness of 10 to 20 nm, a third polymer layer e.g. of PLA with a thickness of 100 nm to 130 nm and a fourth semi-reflecting layer of 5 to 15 nm. The skilled person is aware that a TiO₂ layer will spontaneously form by oxidation if Ti is used for the first reflecting layer. The thickness of the spontaneously formed TiO₂ layer will depend on the thickness of the Ti layer. Typically the spontaneously, native formed TiO₂ layer will be about 50% of the thickness of the Ti layer. Thus, in the specific afore-mentioned set-up, the second transparent TiO₂ layer may form spontaneously from the first reflecting Ti layer. If one additionally wants to include in the specific set-up a fifth transparent layer above the fourth semi-reflecting layers of a thickness of 20 to 30 nm, one will, however, have to actively dispose this layer.

In another set-up, one may use a first reflecting layer of Ti with a thickness of 90 to 110 nm, a second transparent layer of TiO₂ with a thickness of 120 to 140 nm, a third polymer layer e.g. of PLA with a thickness of 90 nm to 110 nm and a fourth semi-reflecting layer of 5 to 15 nm. In this embodiment, the second transparent layer will not spontaneously form, but will have to be actively disposed on the first reflecting layer. If one additionally wants to include in the specific set-up a fifth transparent layer above the fourth semi-reflecting layers of a thickness of 5 to 15 nm, one will have to actively dispose such a layer.

If in the following, reference is made to a polymer layer, this applies to the polymer layer of both, devices in accordance with the first and second aspect of the invention. Thus, the term polymer layer and third polymer layer are used interchangeable herein.

A polymer layer is positioned between the reflecting layers or e.g. the reflecting, transparent and semi-reflecting layers. Said polymer layer is, therefore, not directly exposed to the outside of the device, i.e. it is not positioned on the surface of said device. As set out above, at least the reflecting and/or semi-reflecting layer in contact with the natural product is configured such that biomolecules as defined above are able to penetrate said layer and, therefore, are able to contact the polymer layer. In preferred embodiments of the invention, the polymer layer is transparent.

According to the present invention, the polymer layer may be a biodegradable polymer layer and/or a polymer layer capable of swelling/shrinking and/or a polymer layer capable of absorbing biomolecules. In the following, the polymer layers are described in detail.

The polymer layer of the present invention is made of a polymer. A “polymer” may in general be classified as either being a naturally occurring polymer (e.g. as present in or secreted by microorganisms such as agarose in algae) or a “man-made”, synthetic polymer. A naturally-occurring polymer may also be referred to as “biopolymer”. Thus, a synthetic polymer according to the definition used here cannot be found in nature in exactly the same condition and/or modification and/or conformation. However, every naturally occurring polymer (biopolymer) which has been subjected to modification (such as e.g. cross-linking) resulting in conditions and/or conformations which are not naturally occurring has been transformed into a synthetic polymer and is thus no biopolymer any more by consequence. Thus, a modified biopolymer can be classified as synthetic polymer if its state of modification is not found in nature. Preferably, polymers used in the present invention and described in further detail below belong to the class of synthetic polymers as defined above.

In one embodiment of the present invention, the polymer layer is a biodegradable polymer layer. In a further preferred embodiment, the “biodegradable polymer layer” may be the only layer of the layers comprising the reflecting layers and the biodegradable polymer layer, which is degradable by biomolecules as defined above. Said biomolecules penetrate the reflecting layer and contact the biodegradable polymer layer but do not react with the reflecting layer. In a preferred embodiment of the invention, both reflecting layers are not degradable by said biomolecules. The term “biodegradable” defines that the polymer layer of the present invention is degradable by biomolecules comprising enzymes and/or catabolic metabolites as defined above. Therefore, e.g. enzymes/catabolic metabolites secreted by microorganisms present in foods or enzymes/catabolic metabolites secreted by the natural product itself are capable of degrading said polymer layer. The polymer used for the polymer layer is preferably chosen from the group comprising polylactic acid (PLA), poly-L-lactic acid (PLLA), PLGA, PHB and Polyvinylcaprolactame (PVCL) or any other polymer, which falls under the classification of a polymer degradable by biomolecules as defined above. Preferably, biodegradable synthetic polymers can be used. This may also comprise synthetic polymers of gelatine, agarose, dextrose, lipids, cellulose, starch, chitin, polyhydroxyalkanoates, poly(-caprolactone) (PCL) or PCL-systems, poly(ethylene/butylenes) succinate or poly(ethylene/butylenes) adipate. In case the material of said polymer layer is degraded by enzymes and/or catabolic metabolites, this is accompanied by a change of the thickness of the polymer layer and, therefore, ultimately leads to a change of the colour of the device. In especially preferred embodiments, this change of the thickness is irreversible.

In another preferred embodiment of the invention, the biodegradable polymer layer additionally comprises a cross-linking agent. This cross-linking agent may be a bifunctional agent, such as e.g. diisocyanat, glutardialedhyde or Desmodur (Desmodur 2460 M, Bayer). Desmodur products based on diphenylmethane diisocyanate (MDI), toluene diisocyanate (TDI), hexamethylene diisocyanate (HDI) and isophorone diisocyanate (IPDI) may also be used. The skilled person is aware that the concentration of a cross-linker such as Desmodur may be used to influence the refractive index of the polymer layer. Typically, increasing concentration of the cross-linker can be used to increase the refractive index.

In still another preferred embodiment of the invention, the biodegradable polymer layer additionally comprises a solvent. This solvent may be selected from the group comprising chloroform, chloroform+50% v/v EtAc, toluol and trifluoroethanol (TFE).

In a preferred embodiment of the invention, the biodegradable polymer layer has a thickness of about 5 to about 1000 nm. In one of the more preferred embodiments of the invention, said polymer layer has a thickness of about 50 to about 500 nm. In a further preferred embodiment, the biodegradable polymer layer has a thickness of about 250 nm and in an even further preferred embodiment it has a thickness of about 200 nm, of about 180 nm, of about 150 nm, of about 140 nm, of about 130 nm, of about 120 nm, of about 110 nm, of about 100 nm, of about 90 nm or of about 80 nm. All these values mentioned correspond to the respective thickness prior to the contact with the biomolecules, as this reaction may lead to a degradation of the polymer layer.

For the setup of the system it needs to be understood that the thickness of the biodegradable polymer layer correlates with its degradation time. The thicker the biodegradable polymer layer, the longer it takes the biomolecules described above to degrade said layer. By adjusting the thickness of the biodegradable polymer layer, the sensitivity over time of the device may thus be adjusted. Further by adjusting the thickness of the polymer layer, the colour change upon contact with biomolecules can be adjusted.

In another embodiment of the present invention, the polymer layer is a polymer layer capable of swelling/shrinking. In a preferred embodiment of the invention, the swelling/shrinking is induced by a change of the pH and leads to a change in the thickness of the polymer layer and, therefore, to a change in the colour of the device. The change of pH in the environment of the natural product is caused by certain biomolecules described above comprising ionic molecules. Said biomolecules penetrate the reflecting layer, contact the biodegradable polymer layer, change the local pH and lead to swelling/shrinking of the polymer layer. This process, therefore, may not involve any reactions between the biomolecules and the polymer layer besides absorption and may, therefore, not destroy the polymer layer. Furthermore, the reaction may be reversible. However, for the use of the device according to the present invention as described below in more detail, it is preferred that the swelling/shrinking of the polymer is reversible only after the device has been used according to its purpose. This means that the concentration of biomolecules affecting the local pH is proportional to the degree of swelling/shrinking. As, for example, the ionic strength and the local pH of a packaged natural product are usually not influenced by parameters outside of the packaging, the swelling/shrinking reaction will be a permanent reaction as long as the chemical environment of the natural product does not change, i.e. in this case the opening of the packaging. The polymer for the polymer layer capable of swelling/shrinking is preferably chosen from the group comprising polyacrylic acid derivates and polyvinylpyrrolidone derivates. More preferably it is chosen from the synthetic polymers of acrylic acid-acrylamide copolymers.

In another preferred embodiment of the invention, the polymer layer capable of swelling/shrinking may additionally comprise a cross-linking agent. This cross-linking agent may be a bisazide, such as e.g. Na-4,4′-diacidostilbene-2,2′-disulphonate-tetrahydrate, 2,6-bis-(4-acidobenzylidene-methylcyclohexanone). One may also use Desmodur. The concentration of a cross-linker such as Desmodur may be used to influence the refractive index of the polymer layer. Typically, increasing concentration of the cross-linker can be used to increase the refractive index.

In a preferred embodiment of the invention, said polymer layer capable of swelling/shrinking has a thickness of about 10 to about 1000 nm. In one of the more preferred embodiments of the invention, this polymer layer has a thickness of about 100 to about 600 nm. In a further preferred embodiment, the polymer layer has a thickness of about 300 nm. In a further preferred embodiment, the polymer layer has a thickness of about 250 nm and in an even further preferred embodiment it has a thickness of about 200 nm, of about 180 nm, of about 150 nm, of about 140 nm, of about 130 nm, of about 120 nm, of about 110 nm, of about 100 nm, of about 90 nm or of about 80 nm. All these values mentioned correspond to the respective thickness prior to the contact with the biomolecules, as this reaction may lead to a swelling/shrinking of the polymer layer.

In yet another embodiment of the invention, the polymer layer may be a polymer layer capable of absorbing biomolecules as defined above. In a preferred embodiment, the absorption of said biomolecules may not change the thickness of the polymer layer, but rather leads to a change in the refractive index of the polymer layer. As discussed below, a change in the refractive index of the polymer layer leads to a change in the optical setup of the device and ultimately to a change in the colour of the device. Suitable as polymers in this respect are any polymers which absorb biomolecules as defined above proportional to their concentration. In this way, the higher the concentration of biomolecules, e.g. of enzymes secreted from a microorganism associated with said natural product, and the higher the number of such enzymes penetrating the reflecting layer and contacting the polymer layer, the higher the number of enzymes absorbed by and into the polymer layer. In this way, the absorption reaction is again (as for the polymer layer capable of swelling/shrinking) proportional to the concentration of biomolecules and may preferably be reversible only after the natural product has been analyzed by the device of the invention. In preferred embodiments, the absorption may not lead to the degradation and/or shrinking/swelling of the polymer layer, but rather leads to a change in the refractive index of the polymer layer positioned between the two reflecting layers. Preferred synthetic polymers used in such a setup of the device are vinyl polymers having various side-chain groups, as well as polycondensation products (for example polyesters, polyamides, polyimides, polyurethanes and polyureas). More preferred polymers used are: poly(methyl methacrylate); poly(isodecyl methacrylate); poly(2-ethylhexyl methacrylate-co-styrene); poly(ethylhexyl methacrylate); poly(methyl methacrylate-co-2-ethylhexyl acrylate); poly(methyl metacrylate-co-2-ethylhexyl methacrylate); poly(cyclohexyl acrylate); poly(dodecyl methacrylate); poly(vinyl propionate); poly(benzyl methacrylate-co-2-ethyhexyl methacrylate); poly(ethylhexyl methacrylate-co-glycidyl methacrylate); poly(butyl methacrylate); poly(tetrahydrofufuryl methacrylate).

Furthermore, in a preferred embodiment of the invention, said polymer layer capable of absorbing biomolecules has a thickness of about 3 to about 1000 nm. In one of the more preferred embodiments of the invention, this polymer layer has a thickness of about 80 to about 550 nm. In a further preferred embodiment, the polymer layer has a thickness of about 250 nm and in an even further preferred embodiment it has a thickness of about 200 nm, of about 180 nm, of about 150 nm, of about 140 nm, of about 130 nm, of about 120 nm, of about 110 nm, of about 100 nm, of about 90 nm or of about 80 nm. All these values mentioned correspond to the respective thickness prior to the contact with the biomolecules.

However, the polymer layer of the present invention does not comprise any organosilicon compounds and/or organosilicon compound layers or any other layers which render the surface of the layer strongly hydrophobic.

The optical system of each embodiment of the present invention is based on a Fabry-Perot system. This applies to the first embodiment of the invention or the second embodiment of the invention when the fourth semi-reflecting layer is a continuous layer. In general, the varying transmission function of the Fabry-Perot setup is caused by interference between the multiple reflections of light between the two reflecting layers or the first reflecting and the fourth semi-reflecting layer. Constructive interference occurs if the transmitted beams are in phase corresponding to a transmission maximum. In case the transmitted beams are out of phase, destructive interference occurs corresponding to a transmission minimum. Whether the reflected beams are in or out of phase depends inter alia on the thickness of the layer(s) in between the reflecting layers or in between the first reflecting and the fourth semi-reflecting layer, namely the polymer layer and/or the second transparent layer, and on the refractive index of this polymer layer and/or of the second transparent layer in between said two reflecting layers or in between the first reflecting and the fourth semi-reflecting layer. For the whole optical setup to work as Fabry-Perot interferometer, however, two reflecting layers or a first reflecting and a fourth semi-reflecting layer with the properties set out above are essential. By using e.g. electrically conductive metal layers as reflecting and/or semi-reflecting layers, a present reflection signal is amplified without any disturbance of the reflection signal for example due to self-absorption, which occurs in case e.g. 3D clusters (or “islands”) are used. Furthermore, the interpretation of the signal given can be clearer in a setup used here wherein the reflecting layer not in contact with the natural product has a clear, almost two dimensional separation line/border compared to a signal derived from a device using clusters (or “islands”) at this position due to their three dimensional structure and, therefore, more imprecise line/border. Thus, this latter layer can provide advantages when being continuous. Also, there can be interactions between said clusters or islands, which may influence inter alia light passing through said clusters and change the absorption. This may be based on electrons randomly moving within each single cluster (although there is no connection between the clusters themselves): As this effect occurs in every single cluster, there may be interactions between said cluster in the form of dipole interactions influencing light. Clearly, any material or change in the material between said clusters may also influence e.g. light passing through in terms of the absorption. Therefore, any change in the material in between said clusters may also lead to changes in the resulting colour. Such a conformation is not possible in case of a continuous and conductive layer.

Overall, incident light is reflected by the setup described above and ultimately leads in combination with the other features of the present invention to a specific colour of the device which is visible to the human eye, e.g. a red, white, blue or green colour.

It needs to be understood that the three types of polymer layers described above do not represent necessarily three different types of reactive matrices. In certain embodiments of the invention, they may be combined by the nature of the polymer used. For example, a polymer layer capable of swelling/shrinking may also be capable of absorbing biomolecules. In this case, the change of the colour of the device is caused by a change of the thickness of the reactive polymer layer as well as by a change in the refractive index of the reactive polymer layer caused by the absorption of biomolecules.

As far as the second aspect of the invention is concerned, n2 will typically be in the range of about 1 to about 3, such as about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8 or about 2.9. These values will apply also to n5. The person skilled in the art understands that theses values refer to conditions where the devices are looked at under light visible to the human eye. Thus, the refractive index of TiO₂ is 2.52 at 595 nm and the refractive index of SiN is 2.0 at 595 nm.

As far as the second aspect of the invention is concerned, n1 and/or n4 typically will be in the range of about 0.1 to about 1.5 such as about 0.2, 0.3, 0.4, 0.5, 0.6, 0.7, 0.8, 0.9, 1.1, 1.2, 1.3 or 1.4. The person skilled in the art understands that theses values refer to conditions where the devices are looked at under light visible to the human eye.

As far as the second aspect of the invention is concerned, n3 typically will be in the range of about 0.5 to about 2.5 such as about, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3 or about 2.4. The person skilled in the art understands that theses values refer to conditions where the devices are looked at under light visible to the human eye.

As far as the second aspect of the invention is concerned, the refractive index n2 may be > than the refractive index n1, n3 and n4 by at least about 10%, at least about 15%, at least about 20%, at least about 25%, at least about 30%, at least about 35%, at least about 40%, at least about 45% or at least about 50%. The whole setup of the invention is configured such that a change in the thickness and/or in the refractive index of the reactive polymer matrix layer leads to a change of the colour of the device which is visible to the human eye. The expression “whole setup” comprises for this reason all layers of the device, their material, their distances, their thickness etc.

The thicknesses mentioned in the context of the reflecting layers and the polymer layer thus reflect certain embodiments of the invention. They can also be in a different range as long as the optical setup works, wherein a change in the thickness and/or refractive index of the polymer layer leads to a change of the colour of the device which is visible to the human eye.

The change of the colour of the device according to the invention which is visible to the human eye is, therefore, due to the optical setup of the device and not due to a change of colour of the polymer layer itself.

In a further preferred embodiment of the first aspect of the invention, the device according to the invention comprises two reflecting layers wherein the reflecting layer positioned upon the natural product is either identical to the second reflecting layer or differs from the second reflecting layer with respect to its thickness. In general as set out above, the thickness of the reflecting layer positioned upon the natural product correlates to the sensitivity of the device over time and, therefore, may be adjusted and may be thus be different from the thickness of the second layer. In this preferred embodiment of the first aspect of the invention, both reflecting layers are made of gold films which may be about 5 to 35 nm thick and may be identical or different, but in the range of about 5 to 35 nm. Furthermore, in this preferred embodiment of the invention, the polymer layer is a biodegradable polymer layer as set out above. Its material may be chosen from the group comprising PLA, PLLA, PLGA, PHB and PVCL with, e.g. Desmodur as cross-linking agent. By adjusting the thickness and the cross-linking range of the polymer layer, the sensitivity over time can also be adjusted. In this preferred embodiment, the thickness of the biodegradable polymer layer may vary in the range of about 150 to 300 nm. Therefore, not only varying the thickness of the reflecting layer directly positioned upon the natural product but also varying the thickness and cross-linking range of the biodegradable polymer layer allows adjusting the sensitivity of the device over time. Furthermore, this preferred embodiment of the invention may have any additional layers or features as set out above and below.

Furthermore, in some embodiments of the first aspect of the invention, the device according to the invention may comprise an additional layer between the two reflecting layers apart from the polymer layer wherein said additional layer has a high refractive index. Preferably, said additional layer with a high refractive index may be transparent. This may lead to an even clearer interpretation of the colour resulting from the Fabry-Perot system on the basis of the materials used. The colour of a setup comprising said additional layer with a high refractive index may thus not change to a degree which might lead to misinterpretation when looking at the device from different angles and may thus be clear to interpret. Some polymers used in the present setup as described may have a low refractive index and for such a setup, the additional layer with a high refractive index may be advantageous for the overall interpretation of the resulting colour and, therefore, added to the setup. Such an additional layer with a high refractive index may in one preferred embodiment be TiO₂. It may be positioned on the mirror layer and have a thickness ranging from about 10 nm to about 500 nm, preferably from about 50 nm to about 250 nm and more preferably from about 80 nm to about 120 nm.

In yet another preferred embodiment of the first and second aspect of the invention, the device comprises an additional carrier layer. This carrier layer may be positioned on or above at least one reflecting layer, but on the opposite side of the polymer layer and/or second transparent layer. In certain embodiments, this carrier layer is used to fix the device in the packaging of the natural product. In other preferred embodiments, the carrier layer is useful for the production of the device as set out below. In yet other preferred embodiments, the carrier layer is inert to any reaction with the natural product and allows biomolecules to penetrate to the next layer. The carrier layer may e.g. be in a preferred embodiment a PET-film, on top of which e.g. a gold layer is positioned as mirror layer, followed by the polymer layer and the second reflecting layer or followed by the second transparent layer, the third polymer layer and the fourth semi-reflecting layer. The carrier layer may be part of the preparation-process and may in one embodiment be of the same origin as the packaging material for the natural product. In other preferred embodiment, the additional carrier layer is an inorganic or organic carrier selected from the group comprising polyethylenterephtalate, glass, and the like.

In still another preferred embodiment of the first and second aspect of invention, the device comprises a special type of carrier layer, namely a permeable layer having protective properties. Such a layer may be a hydrogel layer. This hydrogel layer me be positioned on the reflecting layer(s) and/or semi-reflecting layer. In one embodiment, this layer may be in direct contact with the natural product and allow the penetration of biomolecules to the next reflecting layer. The hydrogel layer may act as protection layer for the device and may represent a diffusion layer. The hydrogel layer may preferably be a crosslinked and/or stabilized food-contact compatible polymer layer which is swelling in contact with water and ensures that water is attracted in proximity to the biodegradable layer. The crosslinked hydrogel layer may in a preferred embodiment be a poly-acrylic-acid (PAA) layer. The PAA may have been neutralized with KOH so that it does not change the pH in the microenvironment of the biodegradable layer.

The devices according to the invention may have different forms. In a preferred embodiment of the invention, the device may have the form of a square (FIG. 1). In a further preferred embodiment, the device may be a stripe. Both devices may be integrated into the packaging of e.g. meat (see FIG. 1). Both forms may have an identical surface area, but according to their form they cover different specific areas of e.g. meat. In case of the square, a certain area of meat is covered to a very good extent, whereas in case of a stripe, a broad area of meat is covered to a very good extent. In both cases, this is meant to account for the possibility that the spoilage of meat is not necessarily a homogenous process and, therefore, may start at different areas and at different points of time. By having the form of a stripe reaching from one end of the packaging to the other end or as big square on one spot (see FIG. 1), the inventors try to account for this problem by covering different areas of meat. In other embodiments of the invention, the form of the device may be a circle, rectangle, ellipse or any other suitable form.

It needs to be understood that the devices may be a combination of devices with different properties as set out above. In one embodiment of the invention, a combination or assembly of at least two devices is envisaged. In the following, different preferred embodiments of combinations of two devices exemplary for combinations of at least two devices are described. The different devices may be according to the first and/or second aspect of the invention.

In one of these embodiments, the first and the second device combined with each other differ with regard to the thickness of the reflecting layer or the semi-reflecting directly positioned upon the natural product. Therefore, the thicker the reflecting layer or semi-reflecting layer of the first device, the slower the penetration of biomolecules through said first device.

In another embodiment of these combined devices, the two devices differ from each other with regard to the thickness of the polymer layer. In this embodiment, the polymer layers may be identical with respect to their polymer property and composition, i.e. both may be either a biodegradable polymer layer or a polymer layer capable of swelling/shrinking or a polymer layer capable of absorbing biomolecules. However, a different reaction profile of said two polymer layers with biomolecules as defined above is generated by the difference of the thicknesses of said two polymer layers.

In another preferred embodiment of the invention, the two devices might be identical with regard to their thickness, but differ with regard to their polymer properties and composition. Thus, a first device comprised of a biodegradable polymer layer may be combined with a device comprised of a polymer layer capable of swelling/shrinking. In such an embodiment, the first device may be specific for certain biomolecules capable of degrading said biodegradable polymer layer and, therefore, signal their presence, whereas the second device may be capable of signalling the presence of biomolecules capable of inducing a change in the pH.

Such a combined device, therefore, is capable of signalling the presence of biomolecules of a broad range having different characteristics.

Furthermore, in an also preferred embodiment of the invention, the two devices might differ with regard to more than one feature. Thus, in an exemplary embodiment, the two devices might differ in the thicknesses of the reflecting or semi-reflecting layers positioned upon the natural product as well as in the thickness and/or material/composition of the two polymeric layers, e.g., a biodegradable polymer layer of the first device combined with a polymer layer capable of absorbing biomolecules of the second device.

Of course, in other embodiments, further differences in the composition and/or thickness of the two devices are possible due to the application of the combined device. It is thus possible to combine two devices being different in the thicknesses of the polymer layers and the material/composition of said two polymeric layers (e.g., a biodegradable polymer layer combined with a polymer layer capable of swelling/shrinking) as well as being different in the thicknesses of the reflecting and/or semi-reflecting layers. This difference in the reflecting and/or semi-reflecting layers changes the optical setup of the single devices and, therefore, colours may be adjusted and combined as desired.

However, the device according to the invention may of course be comprised of a combination of more than two devices and thus cover a very broad range of reactivity and sensitivity due to the characteristics of each single device. An example for such a device is the following preferred embodiment wherein biodegradable polymer layers with different cross-linking properties are placed next to each other in such a way, that a range of defined biodegradable polymer layers is formed, e.g. a polymer layer with a cross-linking range of 4.5%, next to 8.0%, next to 12.0%, next to 20.0%. Also, a gradient might be formed ranging from 4.5% to 20% cross-linking. The different biodegradable polymer layers (the gradient, resp.) may be positioned between one set of the two reflecting layers thus corresponding to the first aspect of the invention or in a set-up corresponding to the second aspect of the invention. In such set-ups, each biodegradable polymer layer shows a different reaction kinetic with the biomolecules defined above. In case the cross-linking range is low (for example 4.5%), the polymer layer is destroyed fast associated with an early optical signal, i.e. a colour change in this region. If the cross-linking range is high (for example 20%), the polymer layer is destroyed much later due to higher resistance to enzymatic and/or chemical reactions and the colour change, therefore, occurs later in time in the respective region. With a device built in these set-ups, it is possible to cover different time points/frames of the reaction of biomolecules as defined above and with the biodegradable polymer layer and, ultimately, different stages of the decay/spoilage are monitored. Such a system may also be applied by using swellable/shrinkable layers, absorbing layers and/or a combination of the three kinds of polymer layers as described above.

Furthermore, another example for such a combined device is the following preferred embodiment wherein biodegradable polymer layers with different sensitivities for specific enzymes (obtained by different polymers) are placed next to each other combined with references in such a way, that one can deduce which enzymes are present. Again, the different biodegradable polymer layers may be positioned between one set of the two reflecting layers thus corresponding to the first aspect of the invention or in a set-up corresponding to the second aspect of the invention. In such a setup, specific enzymes and, therefore, for example specific microorganisms secreting unique enzymes may be determined.

The term “reference device” according to the present invention defines a device of a specific colour or a colour range, wherein the colour/colour range is not subjected to a change of colour. To this aim, the reference device is in a preferred embodiment of the invention coloured by any technique known to the person skilled in the art. In another embodiment of the invention, the reference device comprises a polymer layer which is not degradable by biomolecules as mentioned above and therefore does not change its thickness in case it is contacted by said biomolecules. Accordingly, the colour of the reference device after exposure to such biomolecules does not change. Thus, the reference device may have one colour which is identical to the colour of the device according to the invention in case the polymer layer is totally intact. Furthermore, the reference device may have a second colour, which is identical to the device according to the invention in case the biodegradable polymer layer is substantially up to totally degraded by biomolecules as mentioned above. Of course, the same applies for a swellable/shrinkable polymer layer, which has two conditions corresponding to different thicknesses corresponding to different colours covered by said reference device. For a polymer layer capable of absorbing biomolecules, the empty and fully absorbed conditions may be covered by reference devices corresponding to the colours resulting from the different refraction indices. The consumer is able to compare the colour of the device according to the invention to two possible thickness-conditions of the polymer layer of the invention. Of course, the reference device may comprise more than one or two colours for comparison reasons. In an also preferred embodiment, the reference device does not display certain specific colours, but is comprised of a non-degradable polymer layer ranging from the thickness of the device of the invention before any degradation to zero and, therefore, displays a colour range. Again, the consumer may compare the colour of the device according to this invention to said colour range. The reference device may be positioned directly next to the device of the invention. Furthermore, the reference device may inert and, therefore, may not influence the natural product itself.

Furthermore, in other embodiments of the present invention, methods for preparing the aforementioned devices are disclosed. In one embodiment, a reflecting and/or semi-reflecting layer, e.g. a thin gold or a thin titan layer is provided. In an alternative embodiment, Inconnel (by CPFilms, brand name: Lummalloy) is provided as said reflecting layer. Onto such an e.g. reflecting layer, a polymer layer is applied. Optionally, an additional layer with a high refractive index may be applied onto said reflective layer before the polymer layer may then be applied onto said additional layer. One may also apply a transparent layer on the reflecting layer and then apply the polymer layer. The polymer layer may be a biodegradable polymer and/or a polymer capable of swelling/shrinking and/or a polymer capable of absorbing biomolecules. Onto the polymer layer, the second reflecting layer, which may be identical or which may differ from the first reflecting layer with respect to its thickness and/or material/composition or a semi-reflecting layer is applied. In another preferred embodiment, the first reflecting layer is applied onto a carrier layer, already mentioned above, e.g. a PET-film is coated with a thin gold layer. In another embodiment of the invention, a second carrier layer may be applied onto the second reflecting or semi-reflecting layer. This leads in a preferred aspect of the invention to the following layers:
carrier layer
reflecting layer, optionally with refractive index n1
optionally a layer with a high refractive index or with refractive index n2
biodegradable polymer layer, optionally with refractive index n3
reflecting layer or semi-reflecting layer with refractive index n4
optionally a second carrier layer (e.g. a hydrogel layer)

In preferred methods for preparing such devices, the polymer layer may be applied by dip coating or film-printing techniques, such as gravure printing, or by spin coating. Such techniques are routine methods to the skilled person in the art. Any other technique known to the person skilled in the art leading to the application of thin polymer layers onto other layers may also be used. In preferred embodiments, PLA is used as material for the polymer layer. In the methods for preparing the polymer layer, PLA may be used in a concentration (weight/volume) ranging from about 1.5% w/v to about 20% w/v in a suitable solvent, such as chloroform, trifluoroethanole, toluen and the like. The concentration of the cross-linking agent used is also critical for the method of preparing the polymer layer. Desmodur might be used as cross-linking agent in a concentration (volume/volume) ranging from about 0.05% v/v to about 5.0% v/v. In a preferred aspect of the invention, the polymer layer is prepared with about 10% (w/v) PLA in trifluoroethanole and about 0.1 to 1.0, preferably 0.2 to 0.5% (v/v) Desmodur as cross-linking agent by gravure printing. In a further preferred embodiment, polyvinylpyrrolidone (PVP) is used as material for the polymer layer. In the methods for preparing the polymer layer, PVP may be used in a concentration (weight/volume) ranging from about 1% w/v to about 50% w/v in a suitable solvent, such as water, alcohols or organic solvents. The concentration of the cross-linking agent used is also critical for the method of preparing the polymer layer. Na-4,4′-diazidostilbene-2,2-disulphonate-tetrahydrate might be used as cross-linking agent in a concentration (volume/volume) ranging from about 0.001% v/v to about 20% v/v. In a preferred aspect of the invention, the polymer layer is prepared with about 15% (w/v) PVP in water and ethanol and about 5% (v/v) Na-4,4′-diazidostilbene-2,2-disulphonate-tetrahydrate as cross-linking agent by gravure printing and subsequent polymerisation under UV for 2 minutes.

As mentioned above, the devices can also be a combined device with different polymer layers, such as for example polymer layers with different cross-linking properties, next to each other positioned between two reflecting layers. In a preferred method for preparing such a device, these different polymer layers are applied next to each other between only one set of reflecting layers by printing techniques. Using gravure printing, the different polymer and cross-linking solutions are applied in one production step onto said reflecting layer, e.g. a gold film, next to each other followed by the application of the second reflecting layer. Therefore, the production of such a device is easy and cheap.

In preferred methods for preparing such devices, the reflecting layer(s), the semi-reflecting layer, the transparent layer and/or the additional layer with high refractive index may be applied by vacuum coating technologies such as evaporation or sputter coating or chemical reactions, such as direct application of gold nanoparticles through a chemical reaction using the reduction of HAuCl₄. Electrically conductive metal layer films, such as a gold film, may in other preferred embodiments applied by vacuum-coating techniques which are easy to produce and to control. In one preferred embodiment, the reflecting layer is made by sputter coating Inconnel onto a carrier such as a pre-treated or non-treated PET film. In an also preferred embodiment, titan layers are made by sputter coating. Such techniques are routine methods to the skilled person in the art. Any other technique known to the person skilled in the art leading to the application of thin reflecting layers onto other layers may also be used.

In certain embodiments of the invention, a method for preparing a device comprising an additional hydrogel layer as set out above is provided. To this aim, the hydrogel layer is applied directly on top of the reflecting layer on the opposite side of the biodegradable polymer layer, such that the hydrogel layer may be in direct contact with the natural product. The crosslinked hydrogel layer in a preferred embodiment is a Poly-Acrylic-Acid (PAA) Layer which has been applied by thin-film printing.

In further embodiments of the invention, a method for preparing a device comprising an additional layer with high refractive index is provided. Said layer is directly applied onto the first reflecting layer. Said additional layer may be TiO₂ applied by sputter coating.

In other embodiments of the invention, a method for preparing a device comprising an additional step of providing a carrier (or substrate) layer for production is disclosed. Onto said substrate layer, the first reflecting layer is applied. This may in certain embodiments simplify the production of the whole device.

In still other embodiments of the invention, methods for analyzing the age and/or quality of a natural product are provided. In one aspect of the invention, the natural product is directly contacted with the device such that the following set up of layers is present:
carrier layer, e.g. PET film
second reflecting layer, e.g. Au
optionally a layer with high refractive index
polymer layer, e.g. PLA
first reflecting layer, e.g. Au.

The first reflecting layer contacts the natural product, e.g. meat. In this setup, the biomolecules as defined above are able to penetrate the first reflecting layer and contact the polymer layer. The consumer is looking onto the carrier layer and, therefore, can directly see the colour of the device. The carrier layer may be part of the packaging of the natural product or may be a separate carrier, such as a PET-film. In this setup, the carrier layer, if present, is of any translucent material, which can be used with natural products, to allow the passage of incident and reflected light. Therefore, in this setup, the consumer is able to analyze the colour of said device by directly looking at the device which may be integrated into the packaging of the natural product. Said device may thus be integrated into a translucent PET-film used for packaging of the natural product, e.g. meat, and positioned into said PET-film such that the first reflecting layer is in direct contact with the meat and the translucent PET-film is directed towards the outside of the packaging. The two reflecting layers may be identical or may differ from each other with respect to their thickness and/or material/composition.

Method for analyzing the age and/or quality of a natural product in accordance with the invention may comprise the comparison of the colour of the device to the colour of a reference device as defined above. The reference device may have one, two or several fixed colours corresponding to possible colours of the device of the invention as already set out above. In case a biodegradable polymer layer is used and this polymer layer is totally degraded, the device might be e.g. of red colour instead of e.g. green colour in case the polymer layer is not affected at all. The reference device may in this case be comprised of a red and a green colour and, therefore, the consumer is able to compare the colour of the device to the reference-device and determine the age and/or quality of a natural product. If the device according to the present invention corresponds to the red colour of said reference device, the natural product is not intact any more and should not be used according to its purpose. On the other hand, if the device according to the invention corresponds to the green colour of said reference device, the age and/or quality of said natural product is a condition, in which the natural product may be used according to its purpose. As set out before the reference device may display a colour range. In this case, the consumer can compare the colour of the device according to the invention to the colour range of the reference device and determine the age and/or quality of the natural product accordingly.

In other embodiments, the present invention is concerned with the use of devices according to the present invention for the analysis of the age and/or quality of a natural product. Via the degradation of a biodegradable polymer layer by enzymes and/or catabolic metabolites and/or swelling/shrinking of the polymer layer by a change of the pH due to biomolecules and/or absorption of biomolecules into and by the polymer layer, said age and/or quality can be analyzed. The concentration of said biomolecules correlates with the thickness of a biodegradable polymer layer because of the degradation reaction. In case of a swellable/shrinkable polymer, the concentration of said biomolecules correlates also with the thickness of said layer due to swelling. For a polymer layer capable of absorbing biomolecules, there is a correlation between the concentration of the biomolecules and the refractive index because of the absorption event. Therefore, there is a correlation between the presence of e.g. enzymes of microorganisms responsible for spoiling foods and the thickness and/or the refractive index of the polymer layer. The thickness and/or the refractive index in turn correlate with the colour of the device visible to the human eye. Therefore, ultimately, the presence of e.g. microorganisms responsible for spoiling foods may correlate with the colour of said device.

Thus, the devices may be used for analyzing the age and/or quality of a natural product. In case foods are analyzed, the edibility of such foods may be analyzed by the consumer. By adjusting certain parameters like the cross-linking grade and/or the thickness of the reflecting layer which is in contact with the natural product, the sensitivity of said device may be adjusted. In case a biodegradable polymer is used, the velocity by which this layer is degraded depends on its thickness and cross-linking grade as well as on the thickness of the reflecting layer which is in contact with the natural product.

In the following, some embodiments in accordance with the invention are illustrated by way of examples. These examples are, however, not to be construed as limiting.

### EXAMPLES

### Example 1

### Exemplary Use of a Device According to the Invention

In this example, the use of a device according to the invention is described for the analysis of the age and/or quality of packaged meat. FIG. 1A-C illustrates two possible ways (which are, of course, not the only possibilities and thus not limiting): In the left picture, the device according to the invention is placed as stripe, in the right picture as square onto the natural product. In both cases, the device is integrated into the packaging and directly contacts the meat. This means, that the consumer can assess the colour of the device directly from above without opening the packaging. This is possible because of the translucent packaging of the meat, in this case a PET-film. In the left case, an integrated reference device is also depicted.

This reference device is comprised of three different colours corresponding to different quality-conditions of the meat, which would be signalled by the colour of the device according to the invention: green for “ok”, yellow for “limit”, and red for “harmful”. According to the quality and/or age of the meat, the device according to the invention changes its colour: FIG. 1B exemplary shows meat of good quality with a green device according to the invention; FIG. 1C exemplary shows meat of bad quality and a corresponding red device. Of course, different colours in between are also possible. By comparing the colour of the device according to the invention to the reference device, the consumer is able to directly analyze the quality of the product. In case there is no integrated reference device (right picture), the description depicted next to the device according to the invention contains instructions for interpreting the colour of the device.

### Example 2

### Sensor for Detecting Fresh and Spoiled Meat Juice

The following devices according to the invention were prepared as described below and tested for their ability to detect enzymes of fresh and spoiled meat juice.

The general protocol for preparing the devices according to the invention was: Either a CrNi-alloy (InConnel, OD 2,2) or a 35 nm thick Au-layer (Hueck, already prepared on a 36 μm thick PET-foil) was used as first reflective layer. Onto this first layer, a polymer layer of PLA in trifluorethanol with weight-percent as indicated below and Desmodur in trifluorethanol with weight-percent as indicated below was printed (parameters: “feine Druckplatte” by Hueck, “Anpressdruck: Walze+15, Rakelmesser 0”, velocity of 5, followed by an incubation for 10 minutes by 80° C.). Finally, an Au-layer as second reflective layer was sputter-coated at 0.08 mbar onto said polymer layer using different times as indicated below in order to obtain different thicknesses of said second reflective layer.

The devices were then incubated with fresh and spoiled meat juice, which was pipetted onto the second reflecting layer. For this purpose, meat (bought as packaged meat in a supermarket) was homogenised in 20 g portions with 180 ml of a standard peptone-glycerol-buffer (also referred to in the following as buffer). Following the homogenisation, the extract was directly frozen in aliquots of 100 μl at −80° C. corresponding to fresh meat juice. For the preparation of spoiled meat juice, 100 μl aliquots of fresh meat juice were incubated in 0.5 ml tubes for 37° C. for 15 hours and then either used directly or also frozen at −80° C.

For each set of fresh and old meat juice, the titers of microorganisms were determined according to standard microbiological tests and calculated as number of microorganisms/gram meat. Due to the incubation step for the spoiled meat juice, this number was increased in said samples.

Following the tests, fresh and spoiled meat juice was then pipetted onto the devices according to the following scheme:

| | Left: fresh meat juice | Right: spoiled meat juice |
|---|---|---|
| 1 | undiluted | undiluted |
| 2 | 1:2 | 1:2 |
| 3 | 1:4 | 1:4 |
| 4 | 1:8 | 1:8 |
| 5 | 1:16 | 1:16 |
| 6 | 1:32 | 1:32 |
| 7 | Buffer | Buffer |
| 8 | ddH₂O | ddH₂O |

Finally, the devices were incubated in parallel at either 4° C., room temperature or 37° C. for either 4 hours or overnight. Thus, 6 similar devices with identical samples were incubated at mentioned conditions in order to obtain results at these temperatures and incubation times.

The devices were then scanned either with or without parafilm.

The following devices were prepared:

### A) Different Desmodur Concentrations in the Polymer Layer

### Fixed Parameters:

first reflecting layer: InConnel, OD 2,2
polymer layer: 10% PLA in trifluorethanol
second reflecting layer: Au, sputter-coated for 6×30 seconds

### Varied Parameter:

| device | final Desmodur concentration (in %) |
|---|---|
| 1 | 10⁻⁶ |
| 2 | 10⁻⁷ |
| 3 | 10⁻⁸ |
| 4 | 10⁻⁹ |
| 5 | 10⁻¹⁰ |
| 6 | 10⁻¹¹ |
| 7 | 10⁻¹² |
| 8 | 10⁻¹³ |

### Fixed Parameters:

first reflecting layer: 35 nm Au (Hueck-foil)
polymer layer: 10% PLA in trifluorethanol
second reflecting layer: Au, sputter-coated for 6×30 seconds

### Varied Parameter:

| Device | final Desmodur concentration (in %) |
|---|---|
| 9 | 10⁻³ |
| 10 | 10⁻⁴ |
| 11 | 10⁻⁵ |
| 12 | 10⁻⁶ |
| 13 | 10⁻⁷ |

### B) Different Sputter-Coating Times for the Au-Layer on Different %-PLA Layers

### Fixed Parameters:

first reflecting layer: InConnel, OD 2,2
polymer layer: PLA, 0.001% Desmodur in trifluorethanol

### Varied Parameters:

| | final PLA concentration (in | sputter-coating times for |
|---|---|---|
| Device | %) | Au-layer (sec) |
| 14 | 8 | 5 |
| 15 | 8 | 30 |
| 16 | 8 | 45 |
| 17 | 8 | 60 |
| 18 | 8 | 90 |
| 19 | 8 | 120 |
| 20 | 8 | 150 |
| 21 | 10 | 5 |
| 22 | 10 | 30 |
| 23 | 10 | 45 |
| 24 | 10 | 60 |
| 25 | 10 | 90 |
| 26 | 10 | 120 |
| 27 | 10 | 150 |
| 28 | 12 | 5 |
| 29 | 12 | 30 |
| 30 | 12 | 45 |
| 31 | 12 | 60 |
| 32 | 12 | 90 |
| 33 | 12 | 120 |
| 34 | 12 | 150 |

### C) Different Sputter-Coating Times for the Au-Layer on Different Polymer Layers

### Fixed Parameter:

first reflecting layer: InConnel, OD 2,2
polymer layer: PLA, Desmodur in trifluorethanol

### Varied Parameters:

| | | | sputter-coating |
|---|---|---|---|
| | final PLA | final Desmodur | times for Au-layer |
| device | concentration (in %) | concentration (in %) | (sec) |
| 35 | 8 | 10⁻⁴ | 4 × 30 |
| 36 | 8 | 10⁻⁵ | 4 × 30 |
| 37 | 8 | 10⁻⁶ | 4 × 30 |
| 38 | 10 | 10⁻⁴ | 4 × 30 |
| 39 | 10 | 10⁻⁵ | 4 × 30 |
| 40 | 10 | 10⁻⁶ | 4 × 30 |
| 41 | 12 | 10⁻⁴ | 4 × 30 |
| 42 | 12 | 10⁻⁵ | 4 × 30 |
| 43 | 12 | 10⁻⁶ | 4 × 30 |
| 44 | 8 | 10⁻⁴ | 45 |
| 45 | 8 | 10⁻⁵ | 45 |
| 46 | 8 | 10⁻⁶ | 45 |
| 47 | 10 | 10⁻⁴ | 45 |
| 48 | 10 | 10⁻⁵ | 45 |
| 49 | 10 | 10⁻⁶ | 45 |
| 50 | 12 | 10⁻⁴ | 45 |
| 51 | 12 | 10⁻⁵ | 45 |
| 52 | 12 | 10⁻⁶ | 45 |

Exemplary, the results for device 38 are shown in FIG. 2. Clearly, incubation with the meat juice samples results in a change of colour dependent on the incubation time and incubation temperature. Also, the signals for the spoiled meat juice are detected earlier due to the increased concentration of microorganisms therein.

### Example 3

### Differences Between Continuous Metal Layers According to the Invention and “Island-Like”/“Cluster” Metal Layers

In this example, the following experiments were performed in order to demonstrate differences between a continuous metal layer according to the invention and “island-like”/“cluster” metal layers.

A) instruments:
coatings: sputter coater CC8000/9 of CemeCon, Würselen, Germany
conductivity measurements: Multimeter 35XP, Meterman, Eindhoven, Netherlands
reflexion-spectrometry: EPP 2000, StellarNet Inc., Oldsmar, USA
transmission-spectroscopy: V-530 UV/VIS, Jasco GmbH, Groβ-Umstadt, Germany
electron microscopy (EM): LEO 1530 VP Gemini, Zeiss, Oberkochen, Germany
simulations: software “Concise Macleod”, Thin Film Center Inc., Tucson, USA

### B) Experimental Protocols:

Initial Coatings:

The following layers were sputter coated onto a glass slide with dimensions 75×25×1 mm: A titan confinement layer of several nm, an aluminium-layer of about 100 nm and a SiNₓ-layer of about 360 nm.

Copper Coatings onto Said Initial Coatings:

The following Cu-layers were each sputter coated onto initial layers as described above in order to obtain three different Cu-samples:

| Sample-ID | Appr. thickness (nm) | Heating of substrate |
|---|---|---|
| Cu 1 | 9 nm | No |
| Cu 2 | 9 nm | Yes, about 500° C. |
| Cu 3 | 18 nm | Yes, about 500° C. |

Titan Coatings onto Said Initial Coatings:

The following Ti-layers were each sputter coated onto initial layers as described above in order to obtain four different Ti-samples (without heating):

| Sample-ID | Appr. thickness (nm) |
|---|---|
| Ti 1 | 1 |
| Ti 2 | 3 |
| Ti 3 | 11 |
| Ti 4 | 14 |

Conductivity Measurements:

Conductivity of the surfaces of all Ti- and Cu-layers were determined using layers with thicknesses as mentioned above directly sputter coated onto glass slides. Contacts for measurement were placed in a distance of 10 mm from each other. For reference purposes, an appr. 150 nm thick Ti-layer was included in the measurements.
Spectroscopy:

Optical reflexion was determined for the whole setup systems comprising (from the top) a thin metal layer (either a “island”/“cluster” or a continuous layer), the transparent middle-layer SiN_{X} and the metallic aluminium layer.

Transmission was determined for the metal layers with thicknesses as mentioned above directly sputter coated onto glass slides.

EM Pictures:

pictures for determination of the surface morphology were taken in 50.000 times and 200.000 times magnification for samples Cu 1, Cu 2, Cu 3 and Ti 2, also directly sputter coated onto glass slides.

Simulations:

The optical constants n and k determined for the Ti- and Cu-bulk layers as well as text-book values were used for simulations of the metal layers directly on glass slides as well as for the whole layer-systems and compared to the values which were determined experimentally.

### C) Results:

Conductivity of the Layers:

| Sample | Resistance |
|---|---|
| Ti 1 | 100 kΩ |
| Ti 2 | 1 Ω |
| Ti 3 | 400 Ω |
| Ti 4 | 300 Ω |
| Cu 1 | 120 Ω |
| Cu 2 | >20 MΩ |
| Cu 3 | 20 Ω |

The Cu 2 layer is not conductive. As the thickness of this layer is identical to the thickness of the Cu 1 layer, it seems that Cu 2 is comprised of “clusters”. All Ti layers are conductive; the conductivity of the Ti layers decreases with decreasing thickness. Thus, the Ti layers are not comprised of clusters but are rather comprised of continuous layers.

EM Pictures:

FIG. 3 shows the pictures taken of the different layers. FIG. 3A depicts the T±2 layer which is comprised of a continuous, non-structured surface, which is almost two-dimensional. This nicely supports the conductivity-data: The thin T±2 layer is not comprised of clusters, but is a continuous layer. In FIG. 3B, the Cu 2 layer, an even distribution of small clusters of several nm is visible; however, these clusters are not connected. For the thicker Cu 3 layer (FIG. 3C), the particles increase in size to about 40 to 50 nm and are connected with each other. Thus, said layer is conductive. For the Cu 1 layer (FIG. 3D), clouds-like structures are present on a diffuse background. Said background is more random and the structures are connected. Thus, also the Cu 1 layer is conductive.
experimental determination of optical constants and comparison to simulations and pictures of the different samples:

FIG. 4A shows a picture of the different Ti layers sputter coated directly on glass. All samples are semi-transparent; for the thin layer Ti 1, hardly any metal layer is visible.

Transmission values were determined for the layers Ti 2, Ti 3, Cu 1 and Cu 2, each directly sputter coated onto a glass slide with the thicknesses as described above. The transmission spectrum was then compared to a simulated spectrum, wherein the optical constants for thick layers with the optical properties of bulk material are used to simulate the spectrum of layers with defined thicknesses. Only if the metallic characteristics are maintained also for these very thin layers, the simulation is identical to the experimental gained spectrum. As FIGS. 4B and C show, this is indeed the case for the Ti 2 (FIG. 4B) and Ti 3 (FIG. 4C) layers as well as for the Cu 1 (FIG. 4D) layer. However, this is not the case for the Cu 2, the “cluster” layer (FIG. 4D).

The difference between the “cluster” layer Cu 2 and Cu 1 (which have an identical thickness) is also directly visible as can be seen in the picture of these layers (FIG. 4E). Cu 2 appears in a green colour, whereas Cu 1 has the typical brown copper-colour.

Furthermore, the “cluster” layer Cu 2 changes its colour after several days as depicted in FIG. 4F. The other layers, however, are not subject to a colour change.

Optical constants n and k were also determined for a whole layer setup, namely for Ti 3 comprising the following layers: 11 nm Ti, 360 nm SiNₓ, 100 nm Al. Again, the transmission spectrum was compared to a simulation for said system. FIG. 5A shows that the simulation indeed meets the determined parameters.

FIGS. 5B and 5C depict the brilliant colours of the whole layer setups Ti 2, Ti 3 and Ti 4 at two angles.

### Example 4

### Determination of Optical Constants for Different Layers and Simulation of Colours of a Device Using Those Optical Constants

In this example, the following experiments were performed in order to determine certain thicknesses of layers in a whole layer setup resulting in defined colours.

### A) Instruments and Protocols (See Also Above, Example 3):

reflexion-spectrometry: EPP 2000, StellarNet Inc., Oldsmar, USA
transmission-spectroscopy: V-530 UV/VIS, Jasco GmbH, Groβ-Umstadt, Germany
simulations: software “Concise Macleod”, Thin Film Center Inc., Tucson, USA

### B) Results:

the optical constants n and k were experimentally determined for the following layers:
10% PLA with 0.5% Desmodur
10% PLA with 0.2% Desmodur
appr. 30 nm Au-layer
Ti-layer: parameters as determined in example 3, see FIGS. 4B and 4C

FIG. 6A shows the results for the polymer-layers with different concentrations of Desmodur. In green, the index of refraction is shown; clearly, n differs for the two concentrations of Desmodur. The same applies to the extinction coefficient k (shown in red). Thus, the optical constants differ depending on the concentration of cross-linking agent present in the PLA-polymer layer.

In FIG. 6B, the optical constants for the Au-layer are depicted and compared to text-book constants. The colours are as described above. The determined constants correspond to the text-book constants.
simulation of the colours of whole setup systems using different layers with different thicknesses

### a) Comparison of Different Polymer Layers

reflexion-spectra of the following two setups were simulated using the experimentally determined constants:
continuous Au—10% PLA, 0.2% Desmodur—Ti (+native TiO₂-layer)
continuous Au—10% PLA, 0.5% Desmodur—Ti (+native TiO₂-layer)

FIG. 7B shows the results; the setup using 10% PLA, 0.2% Desmodur seems to result in the sharper spectrum and thus the more brilliant colour.

### b) Comparison of Polymer Layers of Different Thickness

reflexion-spectra of the following two setups were simulated using the experimentally determined constants:
continuous Au—10% PLA, 0.2% Desmodur of 530 nm—10.5 nm Ti (+25 nm TiO₂-layer)
continuous Au—10% PLA, 0.2% Desmodur of 440 nm—10.5 nm Ti (+25 nm TiO₂-layer)

FIG. 7B shows the spectra, whereas FIG. 7C shows the colours of said two layers. The setup with the “thick” polymer layer is green, whereas the setup with the “thin” polymer layer is red.

### Example 5

### Effect of an Additional Layer with High Refractive Index

A possible disadvantage of whole layer setups as described above may be the colour “change” which will occur when looking at the setup from different angles in case a polymer layer with a low refractive index is used. FIGS. 5B and 5C indicate this “change” of colours. Such a colour change may be overcome by using very thin polymer layers; however, using polymers with low refractive indices, this may not be possible. A possible alternative way to overcome this colour change may be the use of an additional layer with a high refractive index (as e.g. TiO₂ with n=2.35 (510 nm).

This is exemplary shown in the following experiments. Pictures of two different colour stacks were taken from two different angles, wherein one colour stack contains an additional TiO₂-layer (FIGS. 8A and 8B). Clearly, the colour of the colour stack with this additional layer is almost identical (top) in contrast to the massive colour change of the other colour stack (bottom). This result is nicely supported by the reflexion-spectra as depicted in FIGS. 8C and 8D: The spectra of the stack with the additional layer (FIG. 8D) are only slightly shifted, whereas the spectra of the single colour stack are completely different at the different angles (FIG. 8C).

### Example 6

### Simulation of Colours of a Device with an Additional Layer of High Refractive Index Using Optical Constants

The results of example 5 show that an additional layer with a high refractive index reduces a colour change that may occur depending on different viewing-angles in a layer-setup. Thus, an additional TiO₂-layer was now included into the colour-simulations as described in example 4 using the experimentally obtained optical constants. The following setup was used:

continuous Au—100 nm TiO₂-10% PLA, 0.2% Desmodur, 180 nm—10 nm Ti (+native 25 nm TiO₂-layer)

The reflexion spectra of such a whole layer system at two different angles as depicted in FIG. 9A show a shift of only about 40 nm from the spectrum at 0° to the spectrum at 30°. Thus, by using this additional layer, the colour of the system does not significantly change when looking either directly from the top or from the side at about 30′; it is green (FIG. 9B). By a decrease in the thickness of the polymer layer, however, the colour changes to red/orange as depicted in FIG. 9C.

### Example 7

### Simulation of Colour Change Depending on Thickness of Polymer Layer

First, refractive index values of layers with different composition and thickness were experimentally determined by angle-dependent elipsometry. To this end, the following layers were produced:

First, Au was deposited onto Polyethylenterephtalat (PET) films with a thickness of 50 nm by sputter coating. To this end the PET substrate film was attached to a substrate holder and positioned in a vacuum chamber. The chamber was evacuated to 10³ mbar, an argon plasma was ignited and the deposition process started. The polymer layer of a PLA solution in trifluorethanol with 5% w/v PLA and 0.01% v/v Desmodur was printed via gravure printing on a lab printer (K Printing proofer, R K Print-Coat Instruments Ltd., UK) with a speed of 20 m/min and with a thickness of 124 nm.

A second polymer solution was prepared with 7% w/v PLA and 0.1% v/v Desmodur in trifluorethanol at a printing speed of 120 m/min and with a layer thickness of 145 nm. After printing the film was dried in a drying chamber at 80° C. for 10 min.

For reference measurements the Au layer (thickness 50 nm) without an additional polymer layer was also prepared to determine the optical constants of Au.

Then, the refractive index values were determined using an angle-dependend elipsometry (R. M. A. Azzam and N. M. Bashara, Ellipsometry and Polarized Light, North-Holland, 1977, I. Ohhdal and D. Franta, Ellipsometry of Thin Film Systems, E. Wolf ed., Progress in Optics 41, pp. 181-282, Elsevier, Amsterdam, 2000)

The optical constants of the Au layer are very close to values in literature (Lit: Palik: Handbook of Optical Constants, Band I). Values for n at a 600 nm wavelength are 0.27 and for k 3.18. Optical constants for the PLA 5% w/v and 0.01% v/v Desmodur printed with a speed of 20 m/min are for n at a 600 nm wavelength are 1.45 for n and fork 0.015. Values for the PLA 7% w/v and 0.1% v/v Desmodur mixture printed with a speed of 20 m/min are for n at a 600 nm wavelength are 1.44 for n and for k 0.037.

Reflexion-spectra of the following set-ups were then simulated using the experimentally determined refraction index values with the software “The Consice McLeod”, Thin Film Center Inc., Tucson, USA.

The following set up 1 was simulated using the software The Concise McLeod (at a wavelength of 600 nm):
carrier layer: PET
first reflecting layer: Ti, 67 nm thick
second transparent layer: TiO₂, 15 nm thick
third polymer layer: Polylactic acid 5% and Desmodur 0.01%
fourth semi-reflecting layer: Ti, 10 nm
fifth transparent layer: TiO₂, 25 nm

The thickness of the polymer layer was varied from 120 nm, 100 nm, 80 nm, 60 nm, 40 nm, 20 nm down to 0 nm.

FIG. 10A indicates that the reflectance spectrum and thus the colour appearance of the device changes as the polymer layer becomes thinner as it would happen upon degradation. FIG. 10B visualizes how the colour changes from green for a 120 nm thick polymer layer (top square) to pink for 0 nm polymer layer (bottom square). Such a colour change upon enzymatic degradation of the polymer layer could be easily detected by a consumer.

Then the following set up 2 was simulated:
carrier layer: PET
first reflecting layer: Ti, 100 nm thick
second transparent layer: TiO₂, 130 nm thick
third polymer layer: Polylactic acid 5% and Desmodur 0.01%
fourth semi-reflecting layer: Ti, 10 nm
fifth transparent layer: TiO₂, 10 nm

The thickness of the polymer layer was varied from 100 nm, 90 nm, 80 nm, 70 nm, 60 nm, 50 nm, 40 nm, 30 nm, 25 nm, 20 nm, 15 nm, 10 nm, 5 nm down to 0 nm.

FIG. 11A indicates that the reflectance spectrum and thus the colour appearance of the device changes as the polymer layer becomes thinner as it would happen upon degradation. FIG. 11B visualizes how the colour changes from green for a 100 nm thick polymer layer (top square) to red for 0 nm polymer layer (bottom square). Such a colour change upon enzymatic degradation of the polymer layer could be easily detected by a consumer.

### Example 8

### Simulation of Colour Change Depending on Thickness of Polymer Layer

Then, a simulation was undertaken to analyze how the colours of set up 1 and set up 2 of Example 7 depending on the angle of observation.

Using the simulation software “The Concise McLeod”, simulation was carried out by varying the angle of observation from 0° to 45°. Layer assembly and layer thicknesses were kept constant.

One can clearly see from FIG. 12A (set up 1) and FIG. 12B (set up 2) that the colours for a given polymer thickness do not substantially change upon changing the angle of observation from 0° to 45°. Further, the differences for polymer layers of different thickness can be sufficiently distinguished at all angels of observation.

The thickness of the polymer layer was varied from 120 nm, 100 nm, 80 nm, 60 nm, 40 nm, 20 nm down to 0 nm.

### Example 9

### Preparing Set Ups and Comparison with Simulations

A PET film substrate was coated with 100 nm Ti by sputter coating. On this layer, a layer of SiN with a thickness of 160 nm was sputter coated. Then a polymer solution of PLA 7% w/v with Desmodur 0.1% v/v was printed with a Printer Proofer (K Printing proofer, R K Print-Coat Instruments Ltd., UK) with a speed of 30 m/min on the SiN layer. The resulting thickness was 124 nm after drying for 10 min at 80° C.

Next, the reflection spectra was measured with a EPP 2000 (StellarNet Inc., Oldsmar, USA) and the set-up was scanned with an HP color scanner.

FIG. 13A shows the simulated reflection spectrum while FIG. 13B shows the measured reflection spectrum of the set up. The experimentally tested and simulated reflectivity spectra of this set up are similar. Differences are due to a variation in the simulated and the sputtered SiN layer. The sputtered SiNₓ is a stoichiometric mixture of SiN, SiN2 and SiN3 while the simulation was carried out with SiN2 (n(600 nm)=2.13).

FIG. 14 shows the colours as determined for the simulated and the experimentally tested set up. The resulting colour of the experimentally tested set up is very similar to the simulated set up.

### Example 10

### Preparing Set Ups and Comparison with Simulations

Then, a second Ti layer with a thickness of 10 nm was sputtered on the Polymer layer of the set up of example 9. This set up was also experimentally tested as described above.

FIG. 15A shows the simulated reflection spectrum while FIG. 15B shows the measured reflection spectrum of the set up with the additional Ti layer. The experimentally tested and simulated reflectivity spectra of this set up are again similar.

FIG. 16 shows the colours as determined for the simulated and the experimentally tested set up with the additional Ti layer. The resulting colour of the experimentally tested set up is very similar to the simulated set up. The pinholes in the experimentally tested set up are due to the substrate.

Some embodiments of the invention are:
1. A device comprising at least two reflecting layers and a polymer layer positioned between said two reflecting layers wherein the device is configured in such a way that biomolecules are allowed to penetrate at least one of said reflecting layers in order to contact said polymer layer and wherein the device is configured in such a way that a change in the thickness and/or the refraction index of said polymer layer results in a colour change visible to the human eye.
2. A device according to 1. wherein at least one reflecting layer has a thickness of 1 to 100 nm and said polymer layer has a thickness of 5 to 1000 nm prior to being contacted by said biomolecules.
3. A device according to 1. and 2. wherein at least one reflecting layer comprises a mirror layer made of an electrically conductive material.
4. A device according to 3. wherein the reflecting layer comprises a mirror layer made of an electrically conductive metal or an electrically conductive metal film.
5. A device according to 4. wherein said mirror layer is made of titan or gold or a titan or gold film.
6. A device according to 5. wherein said mirror layer made of titan or gold has a thickness of 10 to 60 nm.
7. A device according to 1. to 6. wherein said two reflecting layers are identical.
8. A device according to 1. to 6. wherein said two reflecting layers differ with respect to their thickness and/or their material/composition.
9. A device according to 1. to 8. wherein the polymer layer comprises a biodegradable polymer and/or a polymer capable of swelling/shrinking and/or a polymer capable of absorbing biomolecules.
10. A device according to 9. wherein said polymer layer is a biodegradable polymer layer degradable by biomolecules comprising enzymes and/or catabolic metabolites.
11. A device according to 9. wherein said polymer layer is a polymer layer capable of swelling/shrinking upon contacting biomolecules comprising ionic molecules.
12. A device according to 9. wherein said polymer layer is a polymer layer capable of absorbing biomolecules comprising enzymes and/or catabolic metabolites.
13. A device according to 9. and 10. wherein said polymer layer is a biodegradable polymer layer selected from the group of polymers comprising PLA, PLGA, PHB and polyvinylcaprolactame.
14. A device according to 9. and 11. wherein said polymer layer is a polymer layer capable of swelling/shrinking selected from the group of polymers comprising polyacrylic acid derivatives and polyvinylpyrrolidone derivatives.
15. A device according to 9. and 12. wherein said polymer layer is a polymer layer capable of absorbing biomolecules selected from the group of polymers comprising vinyl polymers having various side-chain groups and polycondensation products such as polyesters, polyamides, polyimides, polyurethanes and polyureas.
16. A device according to any of 9. to 15. wherein said polymer layer has a thickness of 100 to 500 nm prior to being contacted by said biomolecules.
17. A device according to any of 1. to 16. comprising an additional layer between the two reflecting layers apart form the polymer layer wherein said additional layer has a high refractive index.
18. A device according to 17. wherein said additional layer is made of TiO₂ and has a thickness of 5 to 150 nm.
20. A device according to any of 1. to 18. comprising an additional carrier layer on the reflecting layer(s) being positioned on the opposite of said polymer layer.
21. A device according to any of 1. to 19. wherein the device also comprises a reference device.
22. A method for preparing a device according to any of 1. to 20. wherein said method comprises at least the steps of
   a. Providing a first reflecting layer
   b. Applying a polymer layer onto said first reflecting layer
   c. Applying a second reflecting layer onto said polymer layer.
23. A method according to 21. wherein said method comprises the additional step of applying an additional layer with a high refractive index onto said first reflecting layer before the polymer layer is applied onto said additional layer.
23. A method according to 21. and 22. wherein said method comprises the additional step of providing a carrier and applying said first reflecting layer onto said carrier.
24. A method according to 21. to 23. wherein the polymer layer is applied by dip coating or film-printing.
25. A method according to 21. and 23. wherein the reflecting layers and/or the additional layer with high refractive index are applied by sputter-coating, by evaporation or by chemical reactions.
26. A method for analyzing the age and/or quality of a natural product comprising foods and cosmetical products which comprises the following steps:
   a. Providing a device according to any of 1. to 20.
   b. Contacting said device with said natural product
   c. Determining the colour of said device
   d. Comparing the colour of said device with a reference device
   e. Determining the age and/or quality of said natural product according to this comparison.
27. A method for analyzing the age and/or quality of a natural product according to 26. wherein a reflecting layer of said device is being contacted in step b) with said natural product in such a way that biomolecules are allowed to penetrate this reflecting layer and contact the polymer layer.
28. The use of a device according to any of 1. to 20. for the analysis of the age and/or quality of a natural product comprising foods and cosmetical products.
29. The use of a device according to 28. for the analysis of the age and/or quality of a natural product by detecting microorganisms present in said natural product.
30. The use of a device according to 29. for the analysis of the age and/or quality of a natural product by detecting enzymes and/or catabolic metabolites of microorganisms and/or of the natural product via the degradation of said biodegradable polymer by said enzymes and/or catabolic metabolites and/or via swelling/shrinking of said polymer layer upon contacting said biomolecules and/or absorption of biomolecules by and into said polymer layer.

1-32. (canceled)

33. A device comprising at least
a. a first reflecting layer with a refractive index n1;
b. a second transparent layer with a refractive index n2 positioned above said first reflecting layer;
c. a third polymer with a refractive index n3 positioned above said second transparent layer;
d. a fourth semi-reflecting layer with a refractive index n4 positioned above said third polymer layer;
wherein at least the fourth semi-reflecting layer is permeable for biomolecules to be detected,
wherein the device is configured in such a way that a change in the thickness and/or the refraction index of said third polymer layer results in a colour change visible to the human eye;
wherein the n2 is >n1, n3 and n4; and
wherein the refractive index of said first reflecting and/or said fourth semi-reflecting layer is in the range of 0.15 to 2.0, wherein the refractive index of said third polymer layer is in the range of 1.2 to 1.6 and wherein the refractive index of said second transparent layer is in the range of 1.9 to 3.1.

34. A device according to claim 33, wherein said first reflecting layer and said fourth semi-reflecting layer have a thickness of 1 to 100 nm with the thickness of said fourth reflecting layer being smaller than the thickness of said first reflecting layer, wherein said second transparent layer has a thickness of 1 to 150 nm and wherein said third polymer layer has a thickness of 5 to 1000 nm prior to being contacted by said biomolecules

35. A device according to claim 33, wherein said fourth semi-reflecting is a continuous or discontinuous layer, wherein said discontinuous layer is a nanoparticle layer, and wherein said discontinuous nanoparticle layer comprises island-like structures having a size of 5 to 50 nm in diameter.

36. A device according to claim 33, wherein said third polymer layer comprises one or more polymers selected from the group consisting of a biodegradable polymer, a polymer capable of swelling or shrinking and a polymer capable of absorbing biomolecules.

37. A device according to claim 36, wherein said polymer layer is a biodegradable polymer layer degradable by biomolecules comprising enzymes and/or catabolic metabolites.

38. A device according to claim 36, wherein said polymer layer is a biodegradable polymer layer selected from the group of polymers comprising PLA, PLGA, PHB and polyvinylcaprolactame.

39. A device according to claim 36, wherein said polymer layer has a thickness of 100 to 500 nm prior to being contacted by said biomolecules.

40. A device according to claim 33, wherein said second transparent layer is made from materials selected from the group comprising TiO2, Ta₂O₅, ZnS, CeO2, Nb₂O₅, ZrO₂, ZrO₂⁺, TiO₂, TiO or Ti₃O₅.

41. A device according to claim 33, wherein the refractive index n2 is >than the refractive index n1, n3 and n4 by at least 10%.

42. A device according to claim 33 further comprising an additional fifth transparent layer being positioned above said fourth semi-reflecting layer with a refractive index n5, wherein n5 is >n1, n3 and n4.

43. A device according to claim 33, wherein the device also comprises a reference device.

44. A method for preparing a device according to claim 33, wherein said method comprises at least the steps of
a. providing a first reflecting layer having a refractive n1;
b. applying a second transparent above said first reflecting layer having a refractive n2;
c. applying a third polymer layer above said second transparent layer having a refractive n3;
d. applying a fourth semi-reflecting layer above said third polymer layer having a refractive n4,
wherein n2 is >n1, n3 and n4.

45. A method for analyzing the age and/or quality of a natural product comprising foods and cosmetical products which comprises the following steps:
a. providing a device according to claim 33;
b. contacting said device with said natural product;
c. determining the colour of said device;
d. comparing the colour of said device with a reference device; and
e. determining the age and/or quality of said natural product according to this comparison.

46. A method for analyzing the age and/or quality of a natural product according to claim 45, wherein said fourth reflecting layer of said device is being contacted in step b) with said natural product in such a way that biomolecules are allowed to penetrate this reflecting layer and contact the polymer layer.

47. The use of a device according to claim 33 for the analysis of the age and/or quality of a natural product comprising foods and cosmetical products.
